(19) United States
(12) Patent Application Publication
(10) Pub. No.: US 2006/0188889 A1
(43) Pub. Date: Aug. 24, 2006

(54) Use of differentially expressed nucleic acid sequences as biomarkers for cancer

(76) Inventors: Christopher Burgess, Westwood, MA (US); Susan Myerow, Lexington, MA (US); Arunthathi Thiagalingam, Lexington, MA (US); Peter Maimonis, Westwood, MA (US); Gary Molino, Norfolk, MA (US); Lawrence Burgart, Rochester, MN (US); Lisa Allyn Boardman, Rochester, MN (US); Stephen Thibodeau, Rochester, MN (US); Marcia Lewis, Cohasset, MA (US)

Correspondence Address: SONNENSCHEIN NATH & ROSENTHAL LLP;FOR PAULA EVANS, P.O. BOX 061080, WACKER DRIVE STATION, SEARS TOWER, CHICAGO, IL, 60606-1080, US

(21) Appl. No.: 10/700,439

(22) Filed: Nov. 4, 2003

(51) Int. Cl. C12Q 1/68

(52) U.S. Cl. 435/6

(57) ABSTRACT

The present invention relates to novel marker sequences that are differentially expressed in cancer cells or tissue of a subject with cancerous conditions. The present invention also relates to assays for diagnosis, prognosis, staging, monitoring, therapeutic treatment, and marker sequence related agents including probes, primers, antibodies, and therapeutic compositions.

### FIELD OF THE INVENTION

The present invention relates to methods for diagnosis, prognosis, characterization, management, and therapy of cancer including colon cancer, based on the identification of certain colon cancer-associated differentially expressed marker sequences.

### BACKGROUND OF THE INVENTION

Cancers are the second leading cause of death, next to cardiovascular disease, in the United States. The pathological and molecular mechanisms for cancer initiation and promotion have been revealed after decades of researches. Many genes are involved in the initiation and progression of cancers, including oncogenic and tumor suppressive genes. Multiple factors including genetic, endocrinologic, immunologic, and environmental factors, intertwine in the process of transformation and progression of cancers. The control and cure of cancers remain to be one of the most challenging health care tasks. Particularly, one of the most pressing health issues today is diagnosing, monitoring, and treating cancer.

Colorectal carcinoma is a malignant neoplastic disease. There is a high incidence of colorectal carcinoma in the Western world, particularly in the United States. Tumors of this type often metastasize through lymphatic and vascular channels. Many patients with colorectal carcinoma eventually die from this disease. In fact, it is estimated that 62,000 persons in the United States alone die of colorectal carcinoma annually.

However, if diagnosed early, colon cancer may be treated effectively by surgical removal of the cancerous tissue. Colorectal cancers originate in the colorectal epithelium and typically are not extensively vascularized (and therefore not invasive) during the early stages of development. Colorectal cancer is thought to result from the clonal expansion of a single mutant cell in the epithelial lining of the colon or rectum. The transition to a highly vascularized, invasive and ultimately metastatic cancer which spreads throughout the body commonly takes ten years or longer. If the cancer is detected prior to invasion, surgical removal of the cancerous tissue is an effective cure. However, colorectal cancer is often detected only upon manifestation of clinical symptoms, such as pain and black tarry stool. Generally, such symptoms are present only when the disease is well established, often after metastasis has occurred, and the prognosis for the patient is poor, even after surgical resection of the cancerous tissue. Early detection of colorectal cancer therefore is important in that detection may significantly reduce its morbidity.

Invasive diagnostic methods such as endoscopic examination allow for direct visual identification, removal, and biopsy of potentially cancerous growths such as polyps. Endoscopy is expensive, uncomfortable, inherently risky, and therefore not a practical tool for screening populations to identify those with colorectal cancer. Non-invasive analysis of stool samples for characteristics indicative of the presence of colorectal cancer or precancer is a preferred alternative for early diagnosis, but no known diagnostic methods are available which reliably achieve this goal.

### SUMMARY OF THE INVENTION

The present invention relates to nucleic acid sequences that are differentially expressed in cancer tissue compared to normal tissue, and various methods, reagents and kits for diagnosis, staging, prognosis, monitoring and treatment of cancer, including colon cancer.

In one aspect, the present invention provides methods for determining the expression levels of individual and/or combinations of the differentially expressed marker sequences in a biological sample that are indicative of the presence, or stage of the disease, or the efficacy of therapy. The method comprises contacting said sample with a polynucleotide probe or a polypeptide ligand under conditions effective for said probe or ligand to hybridize specifically to a nucleic acid or a polypeptide in said sample, and detecting the presence or absence of marker sequences. In one embodiment, methods are provided to determine the amounts and/or the differentially expressed levels at which the marker sequences of the present invention are expressed in samples. Such methods can comprise contacting said sample with a polynucleotide probe or a polypeptide ligand under conditions effective for said probe to hybridize specifically to the nucleic acids in said sample, and detecting the amounts or differentially expressed level of the marker sequences. In one preferred embodiment, said polynucleotide probe is a polynucleotide designed to identify one of the marker sequences in Tables 1 and 2. In another preferred embodiment, said polypeptide ligand is an antibody.

In another aspect, the present invention provides probes and primers designed to detect transcripts or genomic sequences corresponding to one or more marker sequences of the present invention. The probes and primers may comprise a portion or all of the sequences listed in SEQ ID NOs: 1-93, or sequences complementary thereto, or sequences which hybridize under stringent conditions to a portion or all of SEQ ID NOs: 1-93.

In another aspect, the present invention provides polypetides encoded by the marker sequences, biologically active portions thereof, and polypetide fragments suitable for use as immunogens to raise antibodies directed against polypeptides of the marker sequences of the present invention.

In another aspect, the present invention provides ligands directed to polypeptides and fragments thereof of the marker sequences of the present invention. Preferably, said polypeptide ligands are antibodies. Antibodies of the invention include, but are not limited to, polyclonal, monoclonal, multispecific, human, humanized, or chimeric antibodies, single chain antibodies, Fab fragments, Fv fragments F(ab′) fragments, fragments produced by a Fab expression library, anti-iodiotypic antibodies, or other epitope binding polypeptide. Preferably, an antibody, useful in the present invention for the detection of the individual marker sequences (and optionally at least one additional colon cancer-specific marker), is a human antibody or fragment thereof, including scFv, Fab, Fab′, F(ab′), Fd, single chain antibody, of Fv. Antibodies, useful in the invention may include a complete heavy or light chain constant region, or a portion thereof, or an absence thereof.

Another aspect of the present invention provides a method of assessing whether a subject is suffering from or at risk of developing cancer including colon cancer by detecting the differential expression of the marker sequences of the present invention. In one embodiment, the diagnostic method comprises determining whether a subject has an abnormal mRNA or cDNA and/or protein level of the marker sequences. The method comprises detecting the expression level of the individual and/or the combinations of the marker sequences in a biological sample obtained from a patient. Specifically, the method comprises:

(1). Providing a nucleic acid probe comprising a nucleotide sequence at least about 8 nucleotides in length, at least about 12 nucleotides in length, preferably at least about 15 nucleotides, more preferably about 25 nucleotides, and most preferably at least about 40 nucleotides, and up to all or nearly all of the coding sequence which is complementary to a portion of the coding sequence of a nucleic acid sequence represented by SEQ ID NOs:1-93, or a sequence complementary thereto;

(2). Obtaining a clinical sample from a patient potentially comprising one or more nucleic acid marker sequences;

(3). Providing a second clinical sample from an individual known to not have colon cancer, or a cancer-free tissue of the same patient;

(4). Contacting the nucleic acid probe under stringent conditions with RNA of each of said first and second clinical samples (e.g., in a Northern blot or in situ hybridization assay); and

(5). Comparing (a) the amount of hybridization of the probe with RNA of the first serum sample, with (b) the amount of hybridization of the probe with RNA of the second clinical sample; wherein a statistically change (e.g., either an increase or a decrease) in the amount of hybridization with the RNA of the first clinical sample as compared to the amount of hybridization with the RNA of the second clinical sample is indicative of the presence of one or more marker sequences in the first clinical sample.

In another embodiment, the diagnostic methods comprise detecting the polypeptides encoded by the marker sequences of the present invention. The assay would include contacting the polypeptides of the test cell or tissue with one or more polypeptide ligands specific for the polypeptides represented by SEQ ID NOs: 94-186, and determining the approximate amount of complex formation by the ligands and polypeptides of the test cell or tissue, wherein a statistically significant difference (either an increase or a decrease) in the amount of the complex formed with the polypeptides of a test cell or tissue as compared to a normal cell or tissue is an indication that the test cell is cancerous or pre-cancerous. In particular, the assay evaluates the level of marker polypeptide in the test cells, and preferably, compares the measured level with marker polypeptide detected in at least one control cell, e.g., a normal cell and/or a transformed cell of known phenotype.

In another aspect, the present invention provides DNA and protein microarrays for detecting the differential expression levels of the marker sequences. In some embodiments, the microarrays comprise at least 2, 3, 4, 5, 6, 7, 8, 9, 10, 11, 12, 13, 14, or 15, or more nucleic acids that are complimentary to at least a portion of the coding sequences of the marker sequences represented by SEQ ID NOs: 1-93. In some embodiments, the microarrays comprise antibodies or antigen-binding fragments thereof, that specifically bind to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 different marker polypeptides encoded by nucleic acids comprising a nucleotide sequence selected from the group consisting of SEQ ID NOs: 1-93. In one embodiment, the probe/primer can comprise a sequence that hybridizes under stringent conditions to at least about 7, preferably 12, preferably about 15, more preferably about 25, 50, 75, 100, 125, 150, 175, 200, 250, 300, 350, or 400, or more consecutive nucleotides of SEQ ID NOs: 1-93 of the present invention. In another embodiment, the probe/primer can comprise a sequence that hybridizes under moderately stringent conditions to at least about 7, preferably 12, preferably about 15, more preferably about 25, 50, 75, 100, 125, 150, 175, 200, 250, 300, 350, or 400, or more consecutive nucleotides of SEQ ID NOs: 1-93 of the present invention.

In another aspect, the present invention provides methods for determining cancer prognosis and stage based on examining the expression levels of the nucleic acid marker sequences and polypeptides using the methods described in the present invention.

In one embodiment, the methods comprise:

(1). detecting in a biological sample of the subject at a first point in time, the expression of one or more nucleic acid sequences comprising one or more nucleic acid sequences selected from the group consisting of SEQ ID NOs: 1-93;

(2). repeating step (a) at a subsequent point in time; and

(3). comparing the expression level detected in steps (a) and (b), wherein a change in the expression level is indicative of progression of cancer or a pre-malignant condition thereof in the subject.

In another embodiment, the methods comprise:

(1). detecting in a biological sample of the subject at a first point in time, the expression of one or more polypeptides comprising one or more polypeptide sequences selected from the group consisting of SEQ ID NOs: 94-186;

(2). repeating step (a) at a subsequent point in time; and

(3). comparing the expression level detected in steps (a) and (b), wherein a change in the expression level is indicative of progression of cancer or a pre-malignant condition thereof in the subject.

In another aspect, the present invention also provides methods that permit the assessment and/or monitoring of patients who will be likely to benefit from both traditional and non-traditional treatments and therapies for cancers, particularly colon cancer. The methods include assessing the levels of one or more of the marker sequences in a biological sample for the purposes of determining the status of a patient's disease an/or the efficacy, reaction, and response to cancer or neoplastic disease treatments or therapies that the patient is undergoing.

The present invention also includes methods of assessing the efficacy of a test composition for inhibiting cancer including colon cancer. The methods comprise comparing expression levels of one or more marker sequences in a first biological sample maintained in the presence of a test composition with the expression levels of the same marker sequences in a second biological sample maintained in the absence of the test composition.

In another aspect, the present invention provides assays for determining compounds that modulate the biological activity of the nucleic acids or the polypeptides encoded by the marker sequences. Methods of identifying compounds generally comprise steps in which a compound is placed in contact with a marker sequence, its transcription product, its translation product, or other target, and determination of whether the compound modulates the marker sequence.

In another aspect, the present invention also provides methods for screening drugs that inhibit cancer including colon cancer. Drug screening is performed by adding a test compound to a sample of cells and monitoring the effect. The screening methods may include both in vitro and in vivo screening of a cell or tissue.

In another aspect, the present invention also provides kits for determining the differential expression levels of the marker sequences of the present invention in a biological sample. Such kits can be used to determine (1) presence or absence of cancer, (2) prognosis and stage of cancer, (3) drugs that inhibit cancer, and (4) treatment for cancer.

### DETAILED DESCRIPTION OF THE INVENTION

I General

The present invention is based, in part, on the identification of marker sequences that are differentially expressed (including both over- and under-expression of the sequences) in various types of humans cells (i.e., cells obtained from a human, cultured human cells, archived or preserved human cells, and in vivo cells) relative to normal (i.e., non-cancerous) human cells. It has been discovered that the level of expression of individual marker sequences and combinations of marker sequences described in the present invention correlates with the presence of cancer or pre-malignant condition in a patient. The expression of one or more marker sequences in human cells can be assessed by detecting the RNA transcripts and/or proteins encoded by the marker sequences. Accordingly, the present invention provides methods for identifying cancer, particularly colon cancer, in an individual by screening for sequences which are over- or under-expressed in cancerous cells relative to the level of expression in normal cells, such as cells from colon tissue. Particularly, the present invention provides a method for the identifying colon cancer in an individual by detecting individual marker sequences and/or combinations of marker sequences in the individual relative to a control expression level of the marker sequences in an individual without cancer. The present invention further provides methods for monitoring the onset, progression, or regression of cancer, particularly colon cancer, in an individual by monitoring the expression level of individual marker sequences and/or combinations of marker sequences in the individual at different points in time. The present invention further provides methods for assessing the efficacy of a therapy for inhibiting cancer, particularly colon cancer in a patient by comparing the expression level of individual marker sequences and/or combinations of marker sequences in the individual prior to and after the therapeutic treatment. The present invention further provides methods for selecting a composition for inhibiting cancer, particularly colon cancer, in a patient by comparing the expression level of individual marker sequences and/or combinations of marker sequences in the presence and absence of the composition. The present invention further provides methods for inhibiting cancer, particularly colon cancer, in a patient by administering to the patient a therapeutic composition, wherein the efficacy of the therapeutic composition is indicated by the change in the expression level of individual marker sequences and/or combinations of marker sequences.

In addition to the above methods, the present invention also provides compositions and various kits for the use in the above methods.

II Definitions

As used herein, the term “differentially expressed” refers to expression levels in a test cell that differ significantly from levels in a reference cell, e.g., mRNA is found at levels at least about 25%, at least about 50% to about 75%, at least about 90% increased or decreased, generally at least about 1.2-fold, at least about 1.5-fold, at least about 2-fold, at least about 5-fold, at least about 10-fold, or at least about 50-fold or more increased or decreased in a cancerous cell when compared with a cell of the same type that is not cancerous. The comparison can be made between two tissues, for example, if one is using in situ hybridization or another assay method that allows some degree of discrimination among cell types in the tissue. The comparison may also be made between cells removed from their tissue source. “Differential expression” refers to both quantitative, as well as qualitative, differences in the genes' temporal and/or cellular expression patterns among, for example, normal and neoplastic tumor cells, and/or among tumor cells which have undergone different tumor progression events.

As used herein, the term “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). “A biological sample” further refers to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. Most often, the sample has been removed from an animal, but the term “biological sample” can also refer to cells or tissue analyzed in vivo, i.e., without removal from animal. Typically, a “biological sample” will contain cells from the animal, but the term can also refer to non-cellular biological material, such as non-cellular fractions of blood, saliva, or urine, that can be used to measure the cancer-associated polynucleotide or polypeptides levels. “A biological sample” further refers to a medium, such as a nutrient broth or gel in which an organism has been propagated, which contains cellular components, such as proteins or nucleic acid molecules.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, the term “change in the expression level” refers to either an increase or a decrease of the expression level in a test sample from the control level by an amount greater than the standard error of the assay employed to assess expression. Preferably, the change is by at least about twice, and more preferably three, four, five or ten times that amount. For increase, the change is determined by comparing the expression level in the test sample to the control level. For decrease, the change is determined by comparing the control level to the expression level in the test sample. Alternatively, the decrease is determined by comparing the expression level in the test sample to the control level and the decrease in the expression level is by at least about 15%, 25%, 30%, 40%, 50%, 65%, 80%, or greater. The term “significant change in the specific binding” refers to either an increase or a decrease from the specific binding in the cancer-free sample by at least about 10%, 20%, 25%, 30%, preferably at least about 40%, 50%, more preferably at least about 60%, 70%, or 90%.

As used herein, the term “expression level of one or more nucleic acid sequences” refers to the amount of mRNA transcribed from the corresponding genes that are present in a biological sample. The expression level can be detected with or without comparison to a level from a control sample or a level expected of a control sample.

As used herein, the term “control expression level of one or more nucleic acid sequences” refers to the amount of mRNA transcribed from the corresponding genes that are present in a biological sample representative of healthy, cancer-free subjects. The term “control expression level” can also refer to an established level of mRNA representative of the cancer-free population, that has been previously established based on measurement from healthy, cancer-free subjects.

As used herein, the term “cancerous cell” or “cancer cell”, used either in the singular or plural form, refers to cells that have undergone a malignant transformation that makes them pathological to the host organism. Malignant transformation is a single- or multi-step process, which involves in part an alteration in the genetic makeup of the cell and/or the gene expression profile. Malignant transformation may occur either spontaneously, or via an event or combination of events such as drug or chemical treatment, radiation, fusion with other cells, Viral infection, or activation or inactivation of particular genes. Malignant transformation may occur in vivo or in vitro, and can if necessary be experimentally induced. Malignant cells may be found within the well-defined tumor mass or may have metastasized to other physical locations. A feature of cancer cells is the tendency to grow in a manner that is uncontrollable by the host, but the pathology associated with a particular cancer cell may take any form. Primary cancer cells (that is, cells obtained from near the site of malignant transformation) can be readily distinguished from non-cancerous cells by well-established pathology techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

As used herein, the term “efficacy” refers to either inhibition to some extent, of cell growth causing or contributing to a cell proliferative disorder, or the inhibition, to some extent, of the production of factors (e.g., growth factors) causing or contributing to a cell proliferative disorder. “A therapeutic efficacy” refers to relief of one or more of the symptoms of a cell proliferative disorder. In reference to the treatment of a cancer, a therapeutic efficacy refers to one or more of the following: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; and/or 5) relieving to some extent one or more of the symptoms associated with the disorder. In reference to the treatment of a cell proliferative disorder other than a cancer, a therapeutic efficacy refers to 1) either inhibition to some extent, of the growth of cells causing the disorder; 2) the inhibition, to some extent, of the production of factors (e.g., growth factors) causing the disorder; and/or 3) relieving to some extent one or more of the symptoms associated with the disorder.

As used herein, the term “detectable label” refers to a composition detectable by spectroscopic, photochemical, biochemical, immunochemical, or chemical means.

As used herein, the term “a polynucleotide probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified on bases (7-deazaguanosine, inosine, etc.) or on sugar moiety. In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “hybridization” refers to any process by which a strand of nucleic acid binds with a complementary strand through base pairing.

As used herein, the term “subject” refers to any human or non-human organism.

As used herein, “individual” refers to a mammal, preferably a human.

As used herein, “detecting” refers to the identification of the presence or absence of a molecule in a sample. Where the molecule to be detected is a polypeptide, the step of detecting can be performed by binding the polypeptide with an antibody that is detectably labeled. A detectable label is a molecule which is capable of generating, either independently, or in response to a stimulus, an observable signal. A detectable label can be, but is not limited to a fluorescent label, a chromogenic label, a luminescent label, or a radioactive label. Methods for “detecting” a label include quantitative and qualitative methods adapted for standard or confocal microscopy, FACS analysis, and those adapted for high throughput methods involving multi-well plates, arrays or microarrays. One of skill in the art can select appropriate filter sets and excitation energy sources for the detection of fluorescent emission from a given fluorescent polypeptide or dye. “Detecting” as used herein can also include the use of multiple antibodies to a polypeptide to be detected, wherein the multiple antibodies bind to different epitopes on the polypeptide to be detected. Antibodies used in this manner can employ two or more detectable labels, and can include, for example a FRET pair. A polypeptide molecule is “detected” according to the present invention when the level of detectable signal is at all greater than the background level of the detectable label, or where the level of measured nucleic acid is at all greater than the level measured in a control sample.

As used herein, “detecting” also refers to detecting the presence of a target nucleic acid molecule (e.g., a nucleic acid molecule encoding the marker sequence) refers to a process wherein the signal generated by a directly or indirectly labeled probe nucleic acid molecule (capable of hybridizing to a target, e.g., a sequence encoding Regla, in a serum sample) is measured or observed. Thus, detection of the probe nucleic acid is directly indicative of the presence, and thus the detection, of a target nucleic acid, such as a sequence encoding a marker sequence. For example, if the detectable label is a fluorescent label, the target nucleic acid is “detected” by observing or measuring the light emitted by the fluorescent label on the probe nucleic acid when it is excited by the appropriate wavelength, or if the detectable label is a fluorescence/quencher pair, the target nucleic acid is “detected” by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is “detected” by, for example, autoradiography. Methods and techniques for “detecting” fluorescent, radioactive, and other chemical labels may be found in Ausubel et al. (1995, Short Protocols in Molecular Biology, 3^{rd} Ed. John Wiley and Sons, Inc.). Alternatively, a nucleic acid may be “indirectly detected” wherein a moiety is attached to a probe nucleic acid which will hybridize with the target, such as an enzyme activity, allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers which are specifically designed to hybridize with a portion of the target nucleic acid sequence. Quantitative amplification methods, such as, but not limited to TaqMan, may also be used to “detect” a target nucleic acid according to the invention. A nucleic acid molecule is “detected” as used herein where the level of nucleic acid measured (such as by quantitative PCR), or the level of detectable signal provided by the detectable label is at all above the background level.

As used herein, “detecting” refers further to the early detection of colorectal cancer in a patient, wherein “early” detection refers to the detection of colorectal cancer at Dukes stage A or preferably, prior to a time when the colorectal cancer is morphologically able to be classified in a particular Dukes stage. “Detecting” as used herein further refers to the detection of colorectal cancer recurrence in an individual, using the same detection criteria as indicated above. “Detecting” as used herein still further refers to the measuring of a change in the degree of colorectal cancer before and/or after treatment with a therapeutic compound. In this case, a change in the degree of colorectal cancer in response to a therapeutic compound refers to an increase or decrease in the expression of the marker sequences including one or more colorectal cancer associated markers, or alternatively, in the amount of the marker polypeptide including one or more colorectal cancer associated markers presented in a clinical sample by at least 10% in response to the presence of a therapeutic compound relative to the expression level in the absence of the therapeutic compound.

As used herein, the term “polypeptide” refers to a polymer in which the monomers are amino acids and are joined together through peptide or disulfide bonds. It also refers to either a full-length naturally-occurring amino acid sequence or a fragment thereof between about 8 and about 500 amino acids in length. Additionally, unnatural amino acids, for example, β-alanine, phenyl glycine and homoarginine may be included. Commonly-encountered amino acids which are not gene-encoded may also be used in the present invention. All of the amino acids used in the present invention may be either the D- or L-optical isomer. The L-isomers are preferred.

As used herein, the term “ligand” refers to any compound that interacts with the ligand binding domain of a receptor and modulate its activity. The term “ligand” also refers to a molecule, such as a peptide or variable segment sequence, that is recognized by a particular receptor. As one of ordinary skill in the art will recognize, a molecule (or macromolecular complex) can be both a receptor and a ligand. In general, the binding partner having a smaller molecular weight is referred to as the ligand and the binding partner having a greater molecular weight is referred to as a receptor. Representative ligands include but are not limited to drugs, drug derivatives, isomers thereof, hormones, polypeptides, nucleotides, and the like.

The term “antibody” refers to the conventional immunoglobulin molecule, as well as fragments thereof which are also specifically reactive with one of the subject polypeptides. Antibodies can be fragmented using conventional techniques and the fragments screened for utility in the same manner as described herein below for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a polypeptide conferred by at least one CDR region of the antibody. In preferred embodiments, the antibodies, the antibody further comprises a label attached thereto and able to be detected, (e.g., the label can be a radioisotope, fluorescent compound, chemiluminescent compound, enzyme, or enzyme co-factor).

The term “monoclonal antibody” refers to an antibody that recognizes only one type of antigen. This type of antibodies is produced by the daughter cells of a single antibody-producing hybridoma.

As used herein, the terms specific “binding” or “specifically binding”, refers to the interaction of an antibody and a protein or peptide. The interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope A, the presence of a protein containing epitope A (or free, unlabeled A) in a reaction containing labeled “A” and the antibody will reduce the amount of labeled A bound to the antibody.

III Identification of Marker Sequences

One aspect of the present invention pertains to identification of differentially expressed marker sequences (either over- or under-expressed) in a biological sample from a patient with cancerous or pre-malignant conditions. In general, the method of identifying the marker sequences involves providing a pool of target nucleic acids (derived from both tumor and normal cells and/or tissue) comprising RNA transcripts of one or more target genes, or nucleic acids derived from the RNA transcripts, hybridizing the nucleic acid sample to one or more probes, and detecting the hybridized nucleic acids and calculating a relative expression level relative to the control expression level of the same nucleic acids. A variety of methods have been employed to achieve this end. They include differential screening of cDNA libraries with selective probes, subtractive hybridization utilizing DNA/DNA hybrids or DNA/RNA hybrids, RNA fingerprinting and differential display (Mather, et al. (1981) Cell 23:369-378; Hedrick et al. (1984) Nature 308:149-153; Davis et al. (1992) Cell 51:987-1000; Welsh et al. (1992) Nucleic Acids Res. 20:4965-4970; and Liang and Pardee (1992) Science 257:967-971). Recently, PCR-coupled subtractive processes have also been reported (Straus and Ausubel (1990) Proc. Natl. Sci. USA 87:1889-1893; Sive and John (1988) Nucleic Acids Res. 16:10937; Wieland et al. (1990) Proc. Natl. Acad. Sci. USA 87:2720-2724; Wang and Brown (1991) Proc. Natl. Acad. Sci. USA 88:11505-11509; Lisitsyn et al. (1993) Science 259:946-951; Zeng et al. (1994) Nucleic Acids Res. 22:4381-4385; Hubank and Schatz (1994) Nucleic Acids Res. 22:5640-5648). Also recently, a microarray technology (DNA chips) developed by Affymetrix (Santa Clara, Calif.) has been used as a powerful tool to simultaneously identify a large number of differentially expressed genes in a biological sample. Each of these methods can be employed in the present invention and is hereby incorporated by reference in entirety.

By using the Affymetrix chips (GeneChip Human Genome U133 Set), the inventors of the present invention identified two clusters of differentially expressed marker sequences that have shown at least a two-fold change (either increase or decrease) in expression level in biological samples from tumor cells and/or tissue, e.g., colon cancer-derived cells and/or tissue, relative to the expression level in samples from normal cells and/or tissue, e.g., normal colon tissue and/or normal non-colon tissue. Table 1 describes 47 marker sequences that are over-expressed (up-regulated) in tumor cells and/or tissue, e.g., colon cancer-derived cells and/or tissue.

TABLE 1
Over-expressed Marker sequences

| | | | | Corresponding | |
|---|---|---|---|---|---|
| SEQ | | | | Protein | Protein |
| ID | Gene Symbol & | Accession | | Accession | SEQ |
| NO | Locus ID | Number | Type | Number | ID NO |
| 1 | KRT23, 25984 | NM_015515 | RNA | NP_056330 | 94 |
| 2 | REG1A, 5967 | NM_002909 | RNA | NP_002900 | 95 |
| 3 | REG1B, 5968 | NM_006507 | RNA | NP_006498 | 96 |
| 4 | DPEP1, 1800 | NM_004413 | RNA | NP_004404 | 97 |
| 5 | IL8, 3576 | NM_000584 | RNA | NP_00575 | 98 |
| 6 | MMP1, 4312 | NM_002421 | RNA | NP_002412 | 99 |
| 7 | MMP7, 4316 | NM_002423 | RNA | NP_002414 | 100 |
| 8 | SSP1, 6696 | NM_000582 | RNA | NP_000573 | 101 |
| 9 | CXCL10, 3627 | NM_001565 | RNA | NP_001556 | 102 |
| 10 | SULF1, 23213 | NM_015170 | RNA | NP_055985 | 103 |
| 11 | COL5A2, 1290 | NM_000393 | RNA | NP_000384 | 104 |
| 12 | CXCL1, 2919 | NM_001511 | RNA | NP_001502 | 105 |
| 13 | CCL18, 6362 | NM_002988 | RNA | NP_002979 | 106 |
| 14 | CDH11, 1009 | NM_001797 | RNA | NP_001788 | 107 |
| 15 | BST2, 684 | NM_004335 | RNA | NP_004326 | 108 |
| 16 | C20orf97, | NM_021158 | RNA | NP_066981 | 109 |
| | 57761 | | | | |
| 17 | THBS2, 7058 | NM_003247 | RNA | NP_003238 | 110 |
| 18 | G1P3, 2537 | NM_022873 | RNA | NP_075011 | 111 |
| 19 | CKTSF1B1, | NM_013372 | RNA | NP_037504 | 112 |
| | 26585 | | | | |
| 20 | MMP9, 4318 | NM_004994 | RNA | NP_004985 | 113 |
| 21 | RAB31, 11031 | NM_006868 | RNA | NP_006859 | 114 |
| 22 | DD96, 10158 | NM_005764 | RNA | NP_005755 | 115 |
| 23 | SUPT4H1, 6827 | NM_003168 | RNA | NP_003159 | 116 |
| 24 | FXYD5, 53827 | NM_014164 | RNA | NP_054883 | 117 |
| 25 | CSPG2, 1462 | NM_004385 | RNA | NP_004376 | 118 |
| 26 | LAPTM4B, | NM_018407 | RNA | NP_060877 | 119 |
| | 55353 | | | | |
| 27 | SOX4, 6659 | NM_003107 | RNA | NP_003098 | 120 |
| 28 | SORD, 6652 | NM_003104 | RNA | NP_003095 | 121 |
| 29 | MMP12, 4321 | NM_002426 | RNA | NP_002417 | 122 |
| 30 | UBD, 10537 | NM_006398 | RNA | NP_006389 | 123 |
| 31 | DKFZp564I1922, | NM_015419 | RNA | NP_056234 | 124 |
| | 25878 | | | | |
| 32 | COL1A1, 1277 | NM_000088 | RNA | NP_000079 | 125 |
| 33 | PLAB, 9518 | NM_004864 | RNA | NP_004855 | 126 |
| 34 | SCD, 6319 | NM_005063 | RNA | NP_005054 | 127 |
| 35 | CCL20, 6364 | NM_004591 | RNA | NP_004582 | 128 |
| 36 | BACE2, 25825 | NM_012105 | RNA | NP_036237 | 129 |
| 37 | GTF3A, 2971 | NM_002097 | RNA | NP_002088 | 130 |
| 38 | C20orf42, | NM_017671 | RNA | NP_060141 | 131 |
| | 55612 | | | | |
| 39 | OSF-2, 10631 | NM_006475 | RNA | NP_006466 | 132 |
| 40 | SPARC, 6678 | NM_003118 | RNA | NP_003109 | 133 |
| 41 | TGFBI, 7045 | NM_000358 | RNA | NP_000349 | 134 |
| 42 | FN1, 2335 | NM_002026 | RNA | NP_002017 | 135 |
| 43 | COL1A2, 1278 | NM_000089 | RNA | NP_000080 | 136 |
| 44 | S100A11, 6282 | NM_005620 | RNA | NP_005611 | 137 |
| 45 | IFITM1, 8519 | NM_003641 | RNA | NP_003632 | 138 |
| 46 | | AF130095 | RNA | AAG35520 | 139 |
| 47 | COL3A1, 1281 | NM_000090 | RNA | NP_000081 | 140 |

Accordingly, the present invention provides marker sequences in Table 1 that are over-expressed by at least about 2 fold, at least about 5 fold, at least about 10 fold, at least about 20 fold, or at least about 50 fold. In one embodiment, the present invention encompasses marker sequences that are over-expressed (up-regulated) in tumor cells and/or tissue, especially in colon cancer cells and/or tissue and/or colon cancer-derived cell lines. In a preferred embodiment, the marker sequences are over-expressed (up-regulated) by at least about 2 fold, at least about 5 fold, at least about 10 fold, at least about 20 fold, or at least about 50 fold.

Table 2 describes 46 marker sequences that are under-expressed (down-regulated) in tumor cells and/or tissue, e.g., colon cancer-derived cells and/or tissue.

TABLE 2
Under-expressed Marker sequences

| | | | | Corresponding | |
|---|---|---|---|---|---|
| SEQ | | | | Protein | Protein |
| ID | Gene Symbol & | Accession | | Accession | SEQ |
| NO | Locus ID | Number | Type | Number | ID NO |
| 48 | GCG, 2641 | NM_002054 | RNA | NP_002045 | 141 |
| 49 | SPINK5, 11005 | NM_006846 | RNA | NP_006837 | 142 |
| 50 | ANPEP, 290 | NM_001150 | RNA | NP_001141 | 143 |
| 51 | AQP8, 343 | NM_001169 | RNA | NP_001160 | 144 |
| 52 | GUCA2B, 2981 | NM_007102 | RNA | NP_009033 | 145 |
| 53 | CLCA4, 22802 | NM_012128 | RNA | NP_036260 | 146 |
| 54 | PRV1, 57126 | NM_020406 | RNA | NP_065139 | 147 |
| 55 | EKI1, 55500 | NM_018638 | RNA | NP_061108 | 148 |
| 56 | FLJ22595, | NM_025047 | RNA | NP_079323 | 149 |
| | 80117 | | | | |
| 57 | UGT2B15 | NM_001076 | RNA | NP_001067 | 150 |
| 58 | CEACAM7, | NM_006890 | RNA | NP_008821 | 151 |
| | 1087 | | | | |
| 59 | CHGA, 1113 | NM_001275 | RNA | NP_001266 | 152 |
| 60 | HPGD, 3248 | NM_000860 | RNA | NP_000851 | 153 |
| 61 | MGC4172, | NM_024308 | RNA | NP_077284 | 154 |
| | 79154 | | | | |
| 62 | CA4, 762 | NM_000717 | RNA | NP_000708 | 155 |
| 63 | IL1R2, 7850 | NM_004633 | RNA | NP_004624 | 156 |
| 64 | FLJ20127, | NM_017678 | RNA | NP_060148 | 157 |
| | 54827 | | | | |
| 65 | MS4A12, 54860 | NM_017716 | RNA | NP_060186 | 158 |
| 66 | EMP1, 2012 | NM_001423 | RNA | NP_001414 | 159 |
| 67 | SLC4A4, 8671 | NM_003759 | RNA | NP_003750 | 160 |
| 68 | ADH1C, 126 | NM_000669 | RNA | NP_000660 | 161 |
| 69 | CEACAM1, 634 | NM_001712 | RNA | NP_001703 | 162 |
| 70 | MAWBP, 64081 | NM_022129 | RNA | NP_071412 | 163 |
| 71 | PCK1, 5105 | NM_002591 | RNA | NP_002582 | 164 |
| 72 | UGT2B17, 7367 | NM_001077 | RNA | NP_001068 | 165 |
| 73 | HSD17B2 | NM_002153 | RNA | NP_002144 | 166 |
| 74 | LOC63928, | NM_022097 | RNA | NP_071380 | 167 |
| | 63928 | | | | |
| 75 | RDHL, 10170 | NM_005771 | RNA | NP_005762 | 168 |
| 76 | GUCA1B, 2979 | NM_002098 | RNA | NP_002089 | 169 |
| 77 | FHL1, 2273 | NM_001449 | RNA | NP_001440 | 170 |
| 78 | ADAMDEC1, | NM_014479 | RNA | NP_055294 | 171 |
| | 27299 | | | | |
| 79 | SPINK4, 27290 | NM_014471 | RNA | NP_055286 | 172 |
| 80 | CA1, 759 | NM_001738 | RNA | NP_001729 | 173 |
| 81 | SGK, 6446 | NM_005627 | RNA | NP_005618 | 174 |
| 82 | CKB, 1152 | NM_001823 | RNA | NP_001814 | 175 |
| 83 | SLC26A2, 1836 | NM_000112 | RNA | NP_000103 | 176 |
| 84 | RNAHP, 11325 | NM_007372 | RNA | NP_031398 | 177 |
| 85 | MUC2, 4583 | NM_002457 | RNA | NP_002448 | 178 |
| 86 | HMGCS2, 3258 | NM_005518 | RNA | NP_005509 | 179 |
| 87 | CLCA1, 1179 | NM_001285 | RNA | NP_001276 | 180 |
| 88 | MT1F, 4494 | NM_005949 | RNA | NP_005940 | 181 |
| 89 | CA2, 760 | NM_000067 | RNA | NP_000058 | 182 |
| 90 | MT1H, 4496 | NM_005951 | RNA | NP_005942 | 183 |
| 91 | MT1G, 4495 | NM_005950 | RNA | NP_005941 | 184 |
| 92 | ZG16, 123887 | NM_152338 | RNA | NP_689551 | 185 |
| 93 | MT1X, 4501 | NM_005952 | RNA | NP_005943 | 186 |

Accordingly, the present invention provides marker sequences in Table 2 that are under-expressed (down-regulated) by at least about 2 fold, at least about 5 fold, at least about 10 fold, at least about 20 fold, or at least about 50 fold. In one embodiment, the present invention encompasses marker sequences that are over-expressed (down-regulated) in tumor cells and/or tissue, especially in colon cancer cells and/or tissue and/or colon cancer-derived cell lines. In a preferred embodiment, the marker sequences are under-expressed (down-regulated) by at least about 2 fold, at least about 5 fold, at least about 10 fold, at least about 20 fold, or at least about 50 fold.

The present invention also encompasses sequences which differ from the marker sequences identified in Tables 1 and 2, but which produce the same phenotypic effect, for example, an allelic variant.

The present invention further encompasses polynucleotides which are at least about 85%, or at least about 90%, or more preferably equal to or greater than about 95% identical to the sequences of the RNA transcripts or cDNAs of the marker sequences. Sequence identity as used herein refers to the proportion of base matches between two nucleic acid sequences or the proportion amino acid matches between two amino acid sequences. When sequence homology is expressed as a percentage, e.g., 50%, the percentage denotes the proportion of matches over the length of sequence from one sequence that is compared to some other sequence.

The identification of marker sequences that are differentially expressed in tumor cells and/or tissue as compared to normal cells and/or tissue, has applications in a number of ways. For example, diagnosis may be done or confirmed by comparing patient samples with the known expression profiles. Similarly, a particular treatment may be evaluated, such evaluation including whether a therapeutic treatment improves the long-term prognosis in a particular patient. Furthermore, the gene expression profiles or individual genes allow screening drug candidates. These methods can also be done at protein level. That is, protein expression levels of the marker sequences associated with the tumor or pre-malignant conditions can be evaluated for diagnostic and prognostic purposes or for screening candidate composition for inhibiting tumors or pre-malignant conditions.

IV Primers and Probes

The nucleic acid sequences of the identified marker sequences that are differentially expressed in tumor cells and/or tissue will further allow for the generation of probes and primers designed to detect transcripts or genomic sequences corresponding to one or more marker sequences of the present invention. The probe/primer is typically used as one or more substantially purified oligonucleotides. The primer/probe may comprise a portion or all of the sequences listed in SEQ ID NOs: 1-93, or sequences complementary thereto, or sequences which hybridize under stringent conditions to a portion or all of SEQ ID NOs: 1-93. In one embodiment, the probe/primer can comprise a sequence that hybridizes under stringent conditions to at least about 7, preferably about 12, preferably about 15, more preferably about 25, 50, 75, 100, 125, 150, 175, 200, 250, 300, 350, or 400, or more consecutive nucleotides of SEQ ID NOs: 1-93 of the present invention. As used herein, the term “hybridizes under stringent conditions” is intended to describe conditions for hybridization and washing under which nucleotide sequences at least about 75% (about 80%, 85%, preferably about 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6× sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, 14:1675-1680 (1996); Breslauer, et al., Proc. Natl. Acad. Sci. USA, 83:3746-3750 (1986); Van Ness, et al., Nucleic Acids Research, 19: 5143-5151 (1991); McGraw, et al., BioTechniques, 8: 674-678 (1990); and Milner, et al., Nature Biotechnology, 15: 537-541 (1997), all expressly incorporated by reference.

In another embodiment, the probe/primer can comprise a sequence that hybridizes under moderately stringent conditions to at least about 7, preferably 12, preferably about 15, more preferably about 25, 50, 75, 100, 125, 150, 175, 200, 250, 300, 350, or 400, or more consecutive nucleotides of SEQ ID NOs: 1-93 of the present invention. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C. to 60° C., 5×SSC, overnight; followed by washing twice at 65° C. for 20 minutes with each of 2×, 0.5×, and 0.2×SSC containing 0.1% SDS. One skilled in the art will understand that the stringency of hybridization can be readily manipulated, such as by altering the salt content of the hybridization solution and/or the temperature at which the hybridization is performed.

In particular, these probes are useful because they provide a method for detecting mutations in wild-type marker sequences of the present invention. Nucleic acid probes which are complementary to a wild-type marker sequence of the present invention and can form mismatches with mutant marker sequences are provided, allowing for detection by enzymatic or chemical cleavage or by shifts in electrophoretic mobility. Likewise, probes based on the subject sequences can be used to detect transcripts or genomic sequences encoding the same or homologous proteins, for use, for example, in prognostic or diagnostic assays.

Nucleic acid probes may be generated using techniques which are well known to those of skill in the art (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987).

In order to measure the hybridization of a nucleic acid probe to a target sequence in a biological sample, the probe is preferably labeled with a detectable label. In preferred embodiments, the probe further comprises a label group attached thereto and able to be detected. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., DynabeadsTM), fluorescent dyes (e.g., fluorescein, texas red, rhodamine, green fluorescent protein, and the like), radiolabels (e.g.,³H, ¹²⁵I, 35S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated into a nucleic acid probe by any of a number of means well known to those of skill in the art. However, in a preferred embodiment, the label is simultaneously incorporated into the probe during an amplification step in the preparation of the probe polynucleotides. Thus, for example, polymerase chain reaction (PCR), or other amplification reaction, with labeled primers or labeled nucleotides will provide a labeled amplification product, and thus a labeled probe.

Alternatively, a label may be added directly to the probe. Means of attaching labels to polynucleotides are well known to those of skill in the art and include, for example nick translation or end-labeling (e.g. with a labeled RNA) and subsequent attachment (ligation) of a polynucleotide linker joining the sample polynucleotide to a label (e.g., a fluorophore).

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013.).

V Polynucleotide Composition

Full-length cDNA molecules comprising the disclosed nucleic acids of the marker sequences, useful for the generation of probes, primers, or for transcription to produce the protein of the marker sequences, or antibodies thereto may be obtained as follows. The nucleic acid sequences of the marker sequences or a portion thereof comprising at least approximately 8, preferably about 12, preferably about 15, preferably about 25, more preferably about 40 nucleotides up to the full length of the sequence of SEQ ID NOs: 1-93, or a sequence complementary thereto, may be used as a hybridization probe to detect hybridizing members of a cDNA library using probe design methods, cloning methods, and clone selection techniques as described in U.S. Pat. No. 5,654,173, “Secreted Proteins and Polynucleotides Encoding Them,” incorporated herein by reference. Libraries of cDNA may be made from selected tissues, such as normal or tumor tissue, or from tissues of a mammal treated with, for example, a pharmaceutical compound. Preferably, the tissue is the same as that used to generate the nucleic acids, as both the nucleic acid and the cDNA represent expressed genes. Alternatively, many cDNA libraries are available commercially. (Sambrook et al., Molecular Cloning: A Laboratory Manual, 2nd Ed. (Cold Spring Harbor Press, Cold Spring Harbor, N.Y. 1989). The choice of cell type for library construction may be made after the identity of the protein encoded by the nucleic acid-related gene is known. This will indicate which tissue and cell types are likely to express the related gene, thereby containing the mRNA for generating the cDNA.

Members of the library that are larger than the nucleic acid, and preferably that contain the whole sequence of the native message, may be obtained. To confirm that the entire cDNA has been obtained, RNA protection experiments may be performed as follows. Hybridization of a full-length cDNA to an mRNA may protect the RNA from RNase degradation. If the cDNA is not full length, then the portions of the mRNA that arc not hybridized may be subject to RNase degradation. This may be assayed, as is known in the art, by changes in electrophoretic mobility on polyacrylamide gels, or by detection of released monoribonucleotides. Sambrook et al., Molecular Cloning: A Laboratory Manual, 2nd Ed. (Cold Spring Harbor Press, Cold Spring Harbor, N.Y. 1989). In order to obtain additional sequences 5′ to the end of a partial cDNA, 5′ RACE (PCR Protocols: A Guide to Methods and Applications (Academic Press, Inc. 1990)) may be performed.

Genomic DNAs of the marker sequences may be isolated using nucleic acids in a manner similar to the isolation of full-length cDNAs. Briefly, the nucleic acids, or portions thereof, may be used as probes to libraries of genomic DNA. Preferably, the library is obtained from the cell type that was used to generate the nucleic acids. Most preferably, the genomic DNA is obtained from the biological material described herein in the Example. Such libraries may be in vectors suitable for carrying large segments of a genome, such as PI or YAC, as described in detail in Sambrook et al., pages 9.4-9.30. In addition, genomic sequences can be isolated from human BAC libraries, which are commercially available from Research Genetics, Inc., Huntville, Ala., USA, for example. In order to obtain additional 5′ or 3′ sequences, chromosome walking may be performed, as described in Sambrook et al., such that adjacent and overlapping fragments of genomic DNA are isolated. These may be mapped and pieced together, as is known in the art, using restriction digestion enzymes and DNA ligase.

Using the nucleic acids of the invention, corresponding full length genes can be isolated using both classical and PCR methods to construct and probe cDNA libraries. Using either method, Northern blots, preferably, may be performed on a number of cell types to determine which cell lines express the gene of interest at the highest rate.

Classical methods of constructing cDNA libraries in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant marker sequences or portions thereof as primers.

PCR methods may be used to amplify the members of a cDNA library that comprise the desired insert. In this case, the desired insert may contain sequence from the full length cDNA that corresponds to the sequence encoding Regla. Such PCR methods include gene trapping and RACE methods.

Gene trapping may entail inserting a member of a cDNA library into a vector. The vector then may be denatured to produce single stranded molecules. Next, a substrate-bound probe, such as biotinylated oligonucleotide, may be used to trap cDNA inserts of interest. Biotinylated probes can be linked to an avidin-bound solid substrate. PCR methods can be used to amplify the trapped cDNA. To trap sequences corresponding to the full length genes, the labeled probe sequence may be based on the nucleic acid of SEQ ID NOs: 1-93, or a sequence complementary thereto. Random primers or primers specific to the library vector can be used to amplify the trapped cDNA. Such gene trapping techniques are described in Gruber et al., PCT WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In preferred embodiments of RACE, a common primer may be designed to anneal to an arbitrary adaptor sequence ligated to cDNA ends (Apte and Siebert, Biotechniques 15:890-893 (1993); Edwards et al., Nuc. Acids Res. 19:5227-5232 (1991)). When a single gene-specific RACE primer is paired with the common primer, preferential amplification of sequences between the single gene specific primer and the common primer occurs. Commercial cDNA pools modified for use in RACE are available.

Once the full-length cDNA or gene is obtained, DNA encoding variants can be prepared by site-directed mutagenesis, described in detail in Sambrook 15.3-15.63. The choice of codon or nucleotide to be replaced can be based on the disclosure herein on optional changes in amino acids to achieve altered protein structure and/or function.

As an alternative method to obtaining DNA or RNA from a biological material, such as serum, nucleic acid comprising nucleotides having the sequence of one or more nucleic acids of the invention can be synthesized. Thus, the invention encompasses nucleic acid molecules ranging in length from about 8 nucleotides (corresponding to at least 12 contiguous nucleotides which hybridize under stringent conditions to or are at least 80% identical to the nucleic acid sequence of SEQ ID NOs: 1-93, or a sequence complementary thereto) up to a maximum length suitable for one or more biological manipulations, including replication and expression, of the nucleic acid molecule. The invention includes but is not limited to (a) nucleic acid comprising the size of the full marker genes, or a sequence complementary thereto; (b) the nucleic acid of(a) also comprising at least one additional gene, operably linked to permit expression of a fusion protein; (c) an expression vector comprising (a) or (b); (d) a plasmid comprising (a) or (b); and (e) a recombinant viral particle comprising (a) or (b).

The sequence of a nucleic acid of the present invention is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired.

In various embodiments described above, the polynucleotides of the present invention can be modified at the base moiety, sugar moiety, or phosphate backbone to improve the stability, hybridization, or solubility of the molecule. For example, detectable markers (avidin, biotin, radioactive elements, fluorescent tags and dyes, energy transfer labels, energy-emitting labels, binding partners, etc.) or moieties which improve hybridization, detection, and/or stability can be attached to the polynucleotides. The polynucleotides can also be attached to solid supports, e.g., nitrocellulose, magnetic or paramagnetic microspheres (e.g., as described in U.S. Pat. Nos. 5,411,863; 5,543,289; for instance, comprising ferromagnetic, super-magnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain recompounds (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

VI Vectors and Host Cells

The present invention further provides vectors and plasmids useful for directing the expression of marker sequences, and further provides host cells which express the vectors and plasmids provided herein. Nucleic acid sequences useful for the expression from a vector or plasmid as described below include, but are not limited to any nucleic acid or gene sequence identified as being differentially regulated by the methods described above, and further include therapeutic nucleic acid molecules, such as antisense molecules. The host cell may be any prokaryotic or eukaryotic cell. Ligating the polynucleotide sequence into a gene construct, such as an expression vector, and transforming or transfecting into hosts, either eukaryotic (yeast, avian, insect or mammalian) or prokaryotic (bacterial cells), are standard procedures well known in the art.

Vectors

There is a wide array of vectors known and available in the art that are useful for the expression of differentially expressed nucleic acid molecules according to the invention. The selection of a particular vector clearly depends upon the intended use the polypeptide encoded by the differentially expressed nucleic acid. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the sequence of interest (e.g., the marker sequences) that permit the transcription and translation of the sequence. Sequences that permit the transcription of the linked sequence of interest include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al.,1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing compound (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) nonmammalian DNA motifs in response to a regulatory agent, and a regulatory sequence that is responsive only to this transcriptional regulator.

Tissue-specific promoters may also be used to advantage in differentially expressed sequence-encoding constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful in the present invention are as follows:

Bowman et al., 1995 Proc. Natl. Acad. Sci. USA 92,12115-12119 describe a brain-specific transferrin promoter; the synapsin I promoter is neuron specific (Schoch et al., 1996 J. Biol. Chem. 271, 3317-3323); the nestin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); and the potassium channel promoter is high-frequency firing neuron specific (Gan et al., 1996 J. Biol. Chem 271, 5859-5865). Any tissue specific transcriptional regulatory sequence known in the art may be used to advantage with a vector encoding a differentially expressed nucleic acid sequence obtained from an animal subjected to pain.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

a. Plasmid Vectors.

Any plasmid vector that allows expression of a coding sequence of interest (e.g., the coding sequence of Reg1α)in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of one or more of the polynucleotides of the invention in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Host Cells

Any cell into which a recombinant vector carrying a gene of interest (e.g., a sequence encoding the marker sequences) may be introduced and wherein the vector is permitted to drive the expression of the peptide encoded by the differentially expressed sequence is useful according to the invention. Any cell in which a differentially expressed molecule of the invention may be expressed and preferably detected is a suitable host, wherein the host cell is preferably a mammalian cell and more preferably a human cell. Vectors suitable for the introduction of nucleic acid sequences to host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells may be primary cultured cells, for example, primary human fibroblasts or keratinocytes, or may be an established cell line, such as NIH3T3, 293T or CHO cells. Further, mammalian cells useful in the present invention may be phenotypically normal or oncogenically transformed. It is assumed that one skilled in the art can readily establish and maintain a chosen host cell type in culture.

Introduction of Vectors to Host Cells.

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of vectors comprising a sequence of interest to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

Following transfection with a vector of the invention, eukaryotic (e.g., human) cells successfully incorporating the construct (intra- or extrachromosomally) may be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening may be used, wherein a negative selection is used to enrich for cells taking up the construct and FACS or fluorescence scanning is used to further enrich for cells expressing differentially expressed polynucleotides or to identify specific clones of cells, respectively. For example, a negative selection with the neomycin analog G418 (Life Technologies, Inc.) may be used to identify cells that have received the vector, and fluorescence scanning may be used to identify those cells or clones of cells that express the vector construct to the greatest extent.

VII Polypeptides

One aspect of the present invention pertains to isolated polypeptides which correspond to individual marker sequences of the present invention, and biologically active portions thereof, as well as polypeptide fragments suitable for use as immunogens to raise antibodies directed against a polypeptide encoded by a nucleic acid marker sequence of the present invention. In one embodiment, the native polypeptide encoded by a marker sequence can be isolated from cells or tissue sources by an appropriate purification scheme using standard protein purification techniques. In another embodiment, polypeptides encoded by a nucleic acid marker sequence of the invention are produced by recombinant DNA techniques. Alternative to recombinant expression, a polypeptide encoded by a nucleic acid marker sequence of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide encoded by a nucleic acid marker sequence of the invention include polypeptides comprising amino acid sequences sufficiently identical to or derived from the amino acid sequence of the protein encoded by the nucleic acid marker sequence (e.g., the amino acid sequence listed in the GenBank and IMAGE Consortium database records described herein), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

The polypeptides may contain amino acid substitutions, deletions or insertions made on the basis of similarity in polarity, charge, solubility, hydrophobicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison, Wis.) and algorithms included in RasMol software (University of Massachusetts, Amherst, Mass.) may be used to help determine which and how many amino acid residues in a particular portion of the protein may be substituted, inserted, or deleted without abolishing biological or immunological activity.

The present invention also provides chimeric or fusion proteins corresponding to a marker sequence of the invention. As used herein, a “chimeric protein” or “fusion protein” comprises all or part (preferably a biologically active part) of a polypeptide encoded by a nucleic acid marker sequence of the invention operably linked to a heterologous polypeptide (i.e., a polypeptide other than the polypeptide encoded by the nucleic acid marker sequence). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide encoded by a nucleic acid marker sequence of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide encoded by a nucleic acid marker sequence of the invention can be removed and replaced with a signal sequence from another protein. For example, the gp67 secretory sequence the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.). A signal sequence can be used to facilitate secretion and isolation of the secreted protein or other proteins of interest.

In addition to recombinant production, proteins or portions thereof may be produced manually, using solid-phase techniques (Stewart et al. (1969) Solid-Phase Peptide Synthesis, W H Freeman, San Francisco, Calif.; Merrifield (1963) J Am Chem Soc 5:2149-2154), or using machines such as the 431A peptide synthesizer (Applied Biosystems (ABI), Foster City, Calif.). Proteins produced by any of the above methods may be used as pharmaceutical compositions to treat disorders associated with null or inadequate expression of the genomic sequence.

VIII Antibodies

Another aspect of the present invention pertains to antibodies directed to polypeptides and fragments thereof of the marker sequences of the present invention. An isolated polypeptide encoded by a nucleic acid marker sequence of the present invention, or fragment thereof, can be used as an immunogen to generate antibodies using standard techniques. Antibodies of the invention include, but are not limited to, polyclonal, monoclonal, multispecific, human, humanized, or chimeric antibodies, single chain antibodies, Fab fragments, Fv fragments F(ab′) fragments, fragments produced by a Fab expression library, anti-iodiotypic antibodies, or other epitope binding polypeptide. Preferably, an antibody, useful in the present invention for the detection of the individual marker sequences (and optionally at least one additional colon cancer-specific marker), is a human antibody or fragment thereof, including scFv, Fab, Fab′, F(ab′), Fd, single chain antibody, of Fv. Antibodies, useful in the invention may include a complete heavy or light chain constant region, or a portion thereof, or an absence thereof. An antibody, useful in the invention, may be obtained from an art recognized host, such as rabbit, mouse, rat, donkey, sheep, goat, guinea pig, camel, horse, or chicken. In one embodiment, an antibody, useful in the invention can be a humanized antibody, in which amino acids have been replaced in the non-antigen binding regions in order to more closely resemble a human antibody, while still retaining the original binding ability. Methods for making humanized antibodies are described in Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400.

Antibodies of the present invention may be monospecific, dispecfic, trispecific, or of greater multispecificity. As such, the individual marker sequences useful for the detection of cancer maybe detected with separate antibodies, or may be detected with the same antibody. Alternatively, a multispecific antibody may exhibit different specificities for different epitopes on the same protein (e.g., different epitopes on a marker sequence). While specificity of an antibody useful in the present invention to one or more additional cancer-specific markers is preferred, antibodies that bind polypeptides with at least 95%, 90%, 85%, 75%, 65%, 55%, and at least 50% identity to a polypeptide useful in the present invention for the detection of cancer, particularly colon cancer are also included in the present invention. Also encompassed in the present invention are antibodies which bind to polypeptide molecules which are encoded by one or more nucleic acid sequences which are complementary to, or hybridize to the sequences of SEQ ID NOs: 1-93.

Antibodies of the present invention which are useful for the detection of colon cancer may further act as agonists or antagonists of the activity of the polypeptide molecules to which they bind, and may thus be useful as therapeutic molecules for the treatment or prevention of colon cancer.

An important, but not limiting, role of an antibody of the present invention is to provide for the purification, or detection of individual marker sequences in a patient sample, including both in vitro and in vivo detection methods. Antibodies useful for the detection of colon cancer as described herein do not have to be used alone, and can be fused to other polypeptides, including a heterologous polypeptide at the N- or C-terminus of the antibody polypeptide sequence. For example, an antibody useful in the present invention may be fused with a detectable label to facilitate detection of the antibody when bound to a target polypeptide. Methods for detectably labeling an antibody polypeptide are known to those of skill in the art.

For the production of antibodies useful in the present invention, various hosts including goats, rabbits, rats, mice, etc., may be immunized by injection with the protein products (or any portion, fragment, or oligonucleotide thereof which retains immunogenic properties) of the candidate genes of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Polyclonal antisera or monoclonal antibodies can be made using methods known in the art. A mammal such as a mouse, hamster, or rabbit, can be immunized with an immunogenic form of a marker polypeptide, fragment, modified form thereof, or variant form thereof. Alternatively, an animal may be immunized with an immunogenic form of one or more additional colon cancer-specific marker polypeptides. Techniques for conferring immunogenicity on such molecules include conjugation to carriers or other techniques well known in the art. For example, the immunogenic molecule can be administered in the presence of adjuvant as described above. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Antibody fragments which can specifically bind to a marker polypeptide of the present invention, or fragments thereof, modified forms thereof, and variants thereof, also may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules include, for example, the antigen binding domain from an antibody of a mouse, rat, or other species, with human constant regions. Standard methods may be used to make chimeric antibodies containing the immunoglobulin variable region which recognizes the gene product of individual marker antigens of the invention (see, e.g., Morrison et al., 1985, Proc. Natl. Acad. Sci. USA 81: 6851; Takeda et al., 1985, Nature 314: 452; U.S. Pat. No. 4,816,567; U.S. Pat. No. 4,816,397).

Antibodies of the invention may be used as therapeutic agents in treating cancers. In a preferred embodiment, completely human antibodies of the invention are used for therapeutic treatment of human cancer patients, particularly those having cervical cancer. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide encoded by a nucleic acid marker sequences of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

An antibody directed against a polypeptide encoded by a nucleic acid marker sequence of the invention (e.g., a monoclonal antibody) can be used to isolate the polypeptide by standard techniques, such as affinity chromatography or immunoprecipitation. Moreover, such an antibody can be used to detect the marker sequence (e.g., in a cellular lysate or cell supernatant) in order to evaluate the level and pattern of expression of the marker sequence. The antibodies can also be used diagnostically to monitor protein levels in tissues or body fluids (e.g. in an ovary-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), sup.bleomycin, mithramycin, and anthramycin (AMC)), and anti-mitotic agents (e.g., vincristine and vinblastine). Alternatively, an antibody can be conjugated to a second antibody to form an antibody heteroconjugate as described in U.S. Pat. No. 4,676,980.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84; Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

IX Detection of the Marker Sequences

In one aspect, the expression levels of the differentially expressed marker sequences are determined in normal and cancer cells and/or tissue, especially the colon cancer cells and/or tissue. In general, the present invention relates to methods of detecting a differentially-expressed nucleic acid sequence in a sample comprising nucleic acid. Such methods can comprise one or more of the following steps in any effective order, e.g., contacting said sample with polynucleotide probes under conditions effective for said probe to hybridize specifically to the nucleic acids of the marker sequences in said sample, and detecting the presence or absence of the nucleic acid marker sequences in said sample. In one preferred embodiment, said probes are polynucleotides designed to identify the marker sequences either in Table 1 or Table 2. The detection method can be applied to any sample, e.g., cultured primary, secondary, or established cell lines, tissue biopsy, blood, urine, stool, cerebral spinal fluid, and other bodily fluids, for any purpose.

In one embodiment, the probes of the individual and/or combinations of the marker sequences are applied to the samples obtained from both the normal and colon cancer cell lines, and the presence of the marker sequences are detected with the methods describes herein. In another embodiment, the probes of the individual and/or combinations of the marker sequences are applied to the samples obtained from both the normal and colon cancer tissue, and the amount of the marker sequences are detected with the methods describes herein. For example, one determination assay can employ the over-expressed marker sequences in combination with an the over-expressed or an under-expressed marker sequences. Moreover, the determination assay can employ a panel of at least two, or at least three, or at least four or more marker sequences, selected from both the over-expressed and the under-expressed marker sequences.

The methods of detecting the presence of the marker sequences can be carried out by any effective process, e.g., by Northern blot analysis, polymerase chain reaction (PCR), reverse transcriptase PCR, RACE PCR, in situ hybridization, etc.. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of the marker polynucleotides, the present invention also relates to determining the amounts at which the marker sequences of the present invention are expressed in samples and determining the differential expression of such marker sequences in samples. Such methods can involve substantially the same steps as described above for presence/absence detection, e.g., contacting with probe, hybridizing, and detecting hybridized probe, but using more quantitative methods and/or comparisons to standards. The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements.

In one embodiment, reverse transcription PCR (RT-PCR) is performed using primers designed to specifically hybridize to a predetermined portion of the marker mRNA sequences isolated from a clinical sample. Generation of a PCR product by such a reaction is thus indicative of the presence of the marker sequences in the sample. The technique of designing primers for PCR amplification is well known in the art. Oligonucleotide primers and probes are about 5 to 100 nucleotides in length, ideally from about 17 to 40 nucleotides, although primers and probes of different length are of use. Primers for amplification are preferably about 17-25 nucleotides. Primers useful according to the invention are also designed to have a particular melting temperature (Tm) by the method of melting temperature estimation. Commercial programs, including Oligo™ (MBI, Cascade, CO), Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50 and 60° C. Preferably, the Tm of a probe useful according to the invention is 7° C. higher than the Tm of the corresponding amplification primers. It is preferred that, following generation of cDNA by RT-PCR, the cDNA fragment is cloned into an appropriate sequencing vector, such as a PCRII vector (TA cloning kit; Invitrogen). The identity of each cloned fragment is then confirmed by sequencing in both directions. It is expected that the sequence obtained from sequencing would be the same as the known sequences of the marker sequences as described herein.

Alternatively, the presence of mRNA sequences encoding the marker sequences may be detected by Northern analysis. Sequence confirmed cDNAs, that is, cDNAs encoding the marker sequences are used to produce ³²P-labeled cDNA probes using techniques well known in the art (see, for example, Ausubel, supra). Labeled probes for Northern analysis may also be produced using commercially available kits (Prime-It Kit, Stratagene, La Jolla, Calif.). Northern analysis of total RNA obtained from a clinical sample may be performed using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at −80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.), wherein the detection of hybridization of a marker-specific probe to the clinical sample is indicative of the presence of the marker sequences and thus may be used to detect cancer such as colon cancer.

In an alternative embodiment, the presence and optionally the quantity of the marker sequences in a clinical sample may be determined using the Taqman™ (Perkin-Elmer, Foster City, Calif.) technique, which is performed with a transcript-specific antisense probe (i.e., a probe capable of specifically hybridizing to a marker sequence). This probe is specific for a marker sequence PCR product and is prepared with a quencher and fluorescent reporter probe complexed to the 5′ end of the oligonucleotide. Different fluorescent markers can be attached to different reporters, allowing for measurement of two products in one reaction (e.g., measurement of the marker sequence). When Taq DNA polymerase is activated, it cleaves off the fluorescent reporters by its 5′-to-3′ nucleolytic activity. The reporters, now free of the quenchers, fluoresce. The color change is proportional to the amount of each specific product and is measured by fluorometer; therefore, the amount of each color can be measured and the RT-PCR product can be quantified. The PCR reactions can be performed in 96 well plates so that samples derived from many individuals can be processed and measured simultaneously. The Taqman™ system has the additional advantage of not requiring gel electrophoresis and allows for quantification when used with a standard curve.

The marker sequence-specific antibodies described above may be used to detect the presence of one or more marker sequences in a biological sample by any method known in the art. The immunoassays which can be used include but are not limited to competitive and non-competitive assay systems using techniques such as western blots, radioimmunoassays, ELISA (enzyme linked immunosorbent assay),“sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e. g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100,1% sodium deoxycholate, 0.1% SDS, 0.15 M NaCI, 0.01 M sodium phosphate at pH 7.2,1% Trasylol) supplemented with protein phosphatase and/or protease inhibitors (e. g., EDTA, PMSF, aprotinin, sodium vanadate), adding the antibody of interest to the cell lysate, incubating for a period of time (e. g., 1-4 hours) at 4 C., adding protein A and/or protein G sepharose beads to the cell lysate, incubating for about an hour or more at 4 C., washing the beads in lysis buffer and resuspending the beads in SDS/sample buffer. In the case of immunonprecipitation of a serum sample, however the above protocol is carried out absent the cell lysis step. The ability of the antibody to immunoprecipitate Regl a or TIMP1 (or other colon cancer marker) antigen can be assessed by, e. g., western blot analysis. The parameters that can be modified to increase the binding of the antibody to an antigen and decrease the background (e. g., preclearing the cell lysate with sepharose beads) are well known to those of skill in the art (Ausubel et al, supra).

The individual and/or the combinations of the marker sequences may be detected in a biological sample obtained from a patient using Western blot analysis. Briefly, Western blot analysis comprises preparing protein samples, electrophoresis of the protein samples in a polyacrylamide gel (e. g., 8%-20% SDS-PAGE), transferring the protein sample from the polyacrylamide gel to a membrane such as nitrocellulose, PVDF or nylon, blocking the membrane in blocking solution (e. g., PBS with 3% BSA or non-fat milk), washing the membrane in washing buffer (e. g., PBS-Tween 20), blocking the membrane with primary antibody (the antibody of interest) diluted in blocking buffer, washing the membrane in washing buffer, blocking the membrane with a secondary antibody (which recognizes the primary antibody, e. g., an antihuman antibody) conjugated to an enzymatic substrate (e. g., horseradish peroxidase or alkaline phosphatase) or radioactive molecule (e. g., 32P or 125I) diluted in blocking buffer, washing the membrane in wash buffer, and detecting the presence of the antigen. Methods for the optimization of such an analysis are well known in the art (Ausubel, et al., supra).

Alternatively, the presence of one or more cancer specific marker sequences in a clinical sample may be detected by ELISA. ELISAs comprise preparing antigen, coating the well of a 96 well microtiter plate (or other suitable container) with the antigen, adding the antibody of interest conjugated to a detectable compound such as an enzymatic substrate (e. g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest, that is, the antibody which will bind to a cancer-specific marker) conjugated to a detectable compound may be added to the well. Further, instead of coating the well with the antigen, the antibody may be coated to the well. In this case, a second antibody conjugated to a detectable compound may be added following the addition of the antigen of interest to the coated well. This method may be modified or optimized according techniques which are known to those of skill in the art.

The binding affinity of an antibody to an antigen and the off-rate of an antibody/antigen interaction can be determined by competitive binding assays. One example of such an assay is a radioimmunoassay comprising the incubation of labeled antigen (e. g., marker labeled with 3H or 125I) with an anti-marker antibody in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e. g., 3H or 125I) in the presence of increasing amounts of an unlabeled second antibody.

Preferably, the above detection assays may be carried out using antibodies to detect the protein product encoded by a nucleic acid having the sequence of SEQ ID NOs:1-93, or a sequence complementary thereto. In addition, the above detection assays may be conducted using one or more antibodies which specifically recognize and bind to at least one cancer-specific marker. Accordingly, in one embodiment, the assay would include contacting the proteins of the test cell with an antibody specific for the gene product of a nucleic acid represented by SEQ ID NO:1-93, or a sequence complementary thereto, and determining the approximate amount of immunocomplex formation by the antibody and the proteins of the test cell, wherein a detection of such an immunocomplex is indicative of the presence of the antigen, and thus, permits the detection of colon cancer.

Immunoassays, useful in the present invention include those described above, and can also include both homogeneous and heterogeneous procedures such as fluorescence polarization immunoassay (FPIA), fluorescence immunoassay (FIA), enzyme immunoassay (EIA), and nephelometric inhibition immunoassay (NIA).

In another embodiment, the level of the encoded polypeptide product, i.e., the polypeptide product encoded by a nucleic acid sequence selected from the group consisting of SEQ ID NO:1-93, or a sequence complementary thereto, in a biological fluid (e.g., blood or urine) of a patient may be determined as a way of monitoring the level of expression of the marker nucleic acid sequence in cells of that patient. Such a method would include the steps of obtaining a sample of a biological sample from the patient, contacting the sample (or proteins from the sample) with an antibody specific for an encoded marker polypeptide, and determining the amount of immune complex formation by the antibody, with the amount of immune complex formation being indicative of the level of the marker encoded polypeptide product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of a hyperproliferative disorder, e.g., colon cancer. The level of the marker polypeptide can be used predictably to evaluate whether a sample of cells contains cells which are, or are predisposed towards becoming, transformed cells. Moreover, the subject method can be used to assess the phenotype of cells which are known to be transformed, the phenotyping results being useful in planning a particular therapeutic regimen. For instance, very high levels of the marker polypeptide in sample cells is a powerful diagnostic and prognostic marker for a cancer, such as colon cancer. The observation of marker polypeptide level can be utilized in decisions regarding, e.g., the use of more aggressive therapies.

X Diagnostic Assays

The determination of a detectable increase or decrease in the expression level of one or more marker sequences in a cancer patient compared to a normal patient provides a means of diagnosing or monitoring the patient's disease status, and/or patient response or benefit to cancer therapy. The present invention provides methods for detecting cancer, or alternatively, determining whether a subject is at risk for developing cancer by detecting the disclosed cancer-specific markers (i.e., the nucleic acid sequences of one or more nucleic acid sequences encoding the cancer specific marker and/or polypeptide sequences of one or more cancer specific markers) for the disease or condition encoded thereby. Examples of cancer include but not limited to, adenocarcinoma, lymphoma, blastoma, melanoma, sarcoma, and leukemia. More particularly, examples of cancer also include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Preferably, the cancers include breast, colon, and lung cancer. In a more preferred embodiment, the cancer is colon cancer, and the marker sequences are the ones comprising a nucleic acid sequence selected from the group consisting of SEQ ID NOs:1-93.

In clinical applications, human tissue samples can be screened for the presence and/or absence of the biomarkers identified herein. Such samples may comprise tissue samples, whole cells, cell lysates, or isolated nucleic acids, including, for example, needle biopsy cores, surgical resection samples, lymph node tissue, plasma, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art. The levels of selected markers detected would be compared with statistically valid groups of metastatic, non-metastatic malignant, benign, or normal colon tissue samples.

In one embodiment, the diagnostic method comprises determining whether a subject has an abnormal mRNA or cDNA and/or protein level of the marker sequences. The method comprises using a nucleic acid probe to determine the expression level of the individual and/or the combinations of the marker sequences in a biological sample obtained from a patient. Specifically, the method comprises:
1. Providing a nucleic acid probe comprising a nucleotide sequence at least about 8 nucleotides in length, at least about 12 nucleotides in length, preferably at least about 15 nucleotides, more preferably about 25 nucleotides, and most preferably at least about 40 nucleotides, and up to all or nearly all of the coding sequence which is complementary to a portion of the coding sequence of a nucleic acid sequence represented by SEQ ID NOs:1-93, or a sequence complementary thereto;
2. Obtaining a clinical sample from a patient potentially comprising one or more nucleic acid marker sequences;
3. Providing a second clinical sample from an individual known to not have colon cancer;
4. Contacting the nucleic acid probe under stringent conditions with RNA of each of said first and second clinical samples (e.g., in a Northern blot or in situ hybridization assay); and
5. Comparing (a) the amount of hybridization of the probe with RNA of the first clinical sample, with (b) the amount of hybridization of the probe with RNA of the second clinical sample; wherein a statistically difference (e.g., by at least 0.5 fold, at least 2 fold, at least 5 fold, at least 20 fold, or at least 50 fold) in the amount of hybridization with the RNA of the first clinical sample as compared to the amount of hybridization with the RNA of the second clinical sample is indicative of the presence of one or more marker sequences in the first clinical sample.

In one embodiment, the method comprises in situ hybridization with a probe derived from a given marker nucleic acid sequence, which nucleic acid sequence is represented by SEQ ID NO:1-93, or a sequence complementary thereto. The method comprises contacting the labeled hybridization probe with a sample of a given type of tissue potentially containing cancerous or pre-cancerous cells as well as normal cells, and determining whether the probe labels some cells of the given tissue type to a degree significantly different (e.g., by at least 0.5 fold, at least 2 fold, at least 5 fold, at least 20 fold, or at least 50 fold) than the degree to which it labels other cells of the same tissue type.

Determining by hybridization whether the target is differentially expressed (e.g., up-regulated or down-regulated) in the sample can also be accomplished by any effective means. For instance, the target's expression pattern in the sample can be compared to its pattern in a known control, such as in a normal tissue, or it can be compared to another target in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal sample, the sample is determined or diagnosed not to contain cancer cells. However, if the ratio is at least 2 fold different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Alternatively, the above diagnostic assays may be carried out using antibodies to detect the polypeptides encoded by the nucleic acid marker sequences, which nucleic acid sequences are represented by SEQ ID NOs:1-93, or a sequence complementary thereto. Preferably, the polypeptides have the sequence of one or more of SEQ ID NOs: 94-186. Accordingly, in one embodiment, the assay would include contacting the polypeptides of the test cell or tissue with one or more antibodies specific for the polypeptides represented by SEQ ID NOs: 94-186, and determining the approximate amount of immunocomplex formation by the antibodies and polypeptides of the test cell or tissue, wherein a statistically significant difference in the amount of the immunocomplex formed with the polypeptides of a test or tissue as compared to a normal cell or tissue is an indication that the test cell is cancerous or pre-cancerous. The term “significant difference” refers to a cell phenotype wherein the cell possesses a changed cellular amount of the marker polypeptide relative to a normal cell of similar tissue origin. For example, a cell may have either more or less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

In one embodiment, the assay is performed as a dot blot assay. The dot blot assay finds particular application where tissue samples are employed as it allows determination of the average amount of the marker polypeptide associated with a single cell by correlating the amount of marker polypeptide in a cell-free extract produced from a predetermined number of cells.

It is well established in the cancer literature that tumor cells of the same type (e.g., breast and/or colon tumor cells) may not show uniformly increased expression of individual oncogenes or uniformly decreased expression of individual tumor suppressor genes. There may also be varying levels of expression of a given marker sequence even between cells of a given type of cancer, further emphasizing the need for reliance on a battery of tests rather than a single test. Accordingly, in one aspect, the invention provides for a battery of tests utilizing a number of probes of the invention, in order to improve the reliability and/or accuracy of the diagnostic test.

XI Arrays

In one aspect, the present invention also provides a method wherein nucleic acid probes are immobilized on a DNA chip in an organized array. Oligonucleotides can be bound to a solid support by a variety of processes, including lithography. These nucleic acid probes comprise a nucleotide sequence at least about 8 nucleotides in length, preferably at least about 12 preferably at least about 15 nucleotides, more preferably at least about 25 nucleotides, and most preferably at least about 40 nucleotides, and up to all or nearly all of a sequence which is complementary to a portion of the coding sequence of a marker nucleic acid sequence represented by SEQ ID NO:1-93 and is differentially expressed in cancer cells, such as colon cancer cells. In some embodiments, the microarrays comprise at least 3, 4, 5, 6, 7, 8, 9, 10, 11, 12, 13, 14, or 15, or more nucleic acids that are complimentary to at least a portion of the coding sequences of the marker sequences comprising a nucleic acid sequence selected from the group consisting of SEQ ID NOs: 1-93. The present invention provides significant advantages over the available tests for various cancers, such as colon cancer, because it increases the reliability of the test by providing an array of nucleic acid markers on a single chip.

The method includes obtaining a biopsy, which is optionally fractionated by cryostat sectioning to enrich tumor cells to about 80% of the total cell population. The DNA or RNA is then extracted, amplified, and analyzed with a DNA chip to determine the presence of absence of the marker nucleic acid sequences.

In one embodiment, the nucleic acid probes are spotted onto a substrate in a two-dimensional matrix or array. Samples of nucleic acids can be labeled and then hybridized to the probes. Double-stranded nucleic acids, comprising the labeled sample nucleic acids bound to probe nucleic acids, can be detected once the unbound portion of the sample is washed away.

The probe nucleic acids can be spotted on substrates including glass, nitrocellulose, etc. The probes can be bound to the substrate by either covalent bonds or by non-specific interactions, such as hydrophobic interactions. The sample nucleic acids can be labeled using radioactive labels, fluorophores, chromophores, etc.

Techniques for constructing arrays and methods of using these arrays are described in EP No. 0 799 897; PCT No. WO 97/292 12; PCT No. WO 97127317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

In another aspect, the present invention also provides a protein microarrays. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. In general, the protein microarrays include antigen-binding ligands such as antibodies or fragments thereof, fixed to a solid substrate, wherein the ligands specifically bind to the polypeptides encoded by the marker sequences of the present invention. In one embodiment, the protein microarrays further include at least one control polypeptide molecule. In some embodiments, the microarray comprises antibodies or antigen-binding fragments thereof, that bind specifically to least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 different polypeptides encoded by nucleic acid molecules comprising a nucleotide sequence selected from the group consisting of SEQ ID NOs: 1-93. In certain embodiment, the antibodies are monoclonal or polyclonal antibodies. In another certain embodiment, the antibodies are chimeric, human, or humanized antibodies. In yet another certain embodiment, the antibodies are single chain antibodies, and the antigen-binding fragments are F(ab′)2, Fab, Fd, or Fv fragments.

The solid microarray substrate may include, but not limited to, glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

XII Prognosis, Staging, and Monitoring of Cancer

In one aspect, the present invention provides methods for determining cancer prognosis and stage based on examining the expression levels of the nucleic acid marker sequences and polypeptides using the methods described in the present invention. If cancer is detected in a subject using a technique other than by determining the expression levels of the marker sequences, then the differential expression level of the marker sequences can be used to determine the prognosis and stage for the subject. As used herein, prognosis refers to the prediction of the probable course and outcome of a disease.

In general, methods used for prognosis or stage of cancer involve comparison of the amount of the marker sequences in a sample of interest with that of a control to detect relative differences in the expression of the marker sequences, wherein the difference can be measured qualitatively and/or quantitatively. For example, the expression levels of one or more marker RNAs or polypeptides can be compared with the expression levels of the same marker RNAs or polypeptides in cancer free or normal samples. Alternatively, the expression levels of one or more marker RNAs or polypeptides can also be compared with the expression levels of the same marker RNAs or polypeptides observed in cancers that are known not to progress. In addition, the expression levels of one or more marker RNAs or polylpeptides can also be compared with the expression levels of the same marker RNAs or polypeptides observed in cancers that are known to progress and/or metastasize.

Also, as used herein, cancer stage refers to the sequence of the events, in which cancer develops and causes symptoms. In addition, staging is a process used to describe how advanced the cancerous state is in patient. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage. Methods of the present invention are useful in assaying the staging of cancer. The staging of cancer can be accomplished by determining the expression levels of one or more marker RNAs or polypeptides to a reference expression levels of the same marker RNAs or polypeptides. The reference expression levels of the marker RNAs or polypeptides can be that from cancer free or healthy or cancer samples, wherein the cancer can be at different stages in development.

The present invention further provides methods of monitoring cancer progression or recurrence by measuring the expression levels of the marker RNAs or polypeptides over the time. In one embodiment, the methods comprise:

(1). detecting in a biological sample of the subject at a first point in time, the expression of one or more nucleic acid sequences comprising one or more nucleic acid sequences selected from the group consisting of SEQ ID NOs: 1-93;

(2). repeating step (a) at a subsequent point in time; and

(3). comparing the expression level detected in steps (a) and (b), wherein a change in the expression level is indicative of progression of cancer or a pre-malignant condition thereof in the subject.

In another embodiment, the methods comprise:

(1). detecting in a biological sample of the subject at a first point in time, the expression of one or more polypeptides comprising one or more polypeptide sequences selected from the group consisting of SEQ ID NOs: 94-186;

(2). repeating step (a) at a subsequent point in time; and

(3). comparing the expression level detected in steps (a) and (b), wherein a change in the expression level is indicative of progression of cancer or a pre-malignant condition thereof in the subject.

For example, elevated expression levels of one or more over-expressed marker RNAs or polypeptides, or reduced expression levels of one or more under-expressed marker RNAs or polypeptides in a subsequent point in time relative to an earlier point in time, indicate that the cancer is progressing to a more severe stage. On the other hand, reduced expression levels of one or more over-expressed marker RNAs or polypeptides, or elevated expression levels of one or more under-expressed marker RNAs or polypeptides in a subsequent point in time relative to an earlier point in time, indicate that the cancer is not progressing or is progressing slowly.

The methods used in prognosis, staging, and monitoring cancer can be applied to various types of cancer. Examples of cancer include but not limited to, adenocarcinoma, lymphoma, blastoma, melanoma, sarcoma, and leukemia. More particularly, examples of cancer also include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Preferably, the cancers include breast, colon, and lung cancer. More preferably, the cancer is colon cancer, and the marker sequences are the ones comprising a nucleic acid sequence selected from the group consisting of SEQ ID NOs: 1-93.

XIII Efficacy of Therapy and Therapeutic Compositions

In one aspect, the present invention also provides methods that permit the assessment and/or monitoring of patients who will be likely to benefit from both traditional and non-traditional treatments and therapies for cancers, particularly colon cancer. The present invention thus embraces testing, screening and monitoring of patients undergoing anti-cancer treatments and therapies, used alone, in combination with each other, and/or in combination with anti-cancer drugs, anti-neoplastic agents, chemotherapeutics and/or radiation and/or surgery, to treat cancer patients.

An advantage of the present invention is the ability to monitor, or screen over time, those patients who can benefit from one, or several, of the available cancer therapies, and preferably, to monitor patients receiving a particular type of therapy, or a combination therapy, over time to determine how the patient is faring from the treatment(s), if a change, alteration, or cessation of treatment is warranted; if the patient's disease has been reduced, ameliorated, or lessened; or if the patient's disease state or stage has progressed, or become metastatic or invasive. The cancer treatments embraced herein also include surgeries to remove or reduce in size a tumor, or tumor burden, in a patient. Accordingly, the methods of the invention are useful to monitor patient progress and disease status post-surgery.

The identification of the correct patients for a cancer therapy according to this invention can provide an increase in the efficacy of the treatment and can avoid subjecting a patient to unwanted and life-threatening side effects of the therapy. By the same token, the ability to monitor a patient undergoing a course of therapy using the methods of the present invention can determine whether a patient is adequately responding to therapy over time, to determine if dosage or amount or mode of delivery should be altered or adjusted, and to ascertain if a patient is improving during therapy, or is regressing or is entering a more severe or advanced stage of disease, including invasion or metastasis, as discussed further herein.

A method of monitoring according to this invention reflects the serial, or sequential, testing or analysis of a cancer patient by testing or analyzing the patient's body fluid sample over a period of time, such as during the course of treatment or therapy, or during the course of the patient's disease. For instance, in serial testing, the same patient provides a body fluid sample, e.g., serum or plasma, or has sample taken, for the purpose of observing, checking, or examining the expression levels of one or more of the markers (RNA or polypeptide) of the invention in the patient by measuring the levels of one or more of these markers during the course of treatment, and/or during the course of the disease, according to the methods of the invention.

Similarly, a patient can be screened over time to assess the levels of one or more of the markers in a biological sample for the purposes of determining the status of his or her disease and/or the efficacy, reaction, and response to cancer or neoplastic disease treatments or therapies that he or she is undergoing. It will be appreciated that one or more pretreatment sample(s) is/are optimally taken from a patient prior to a course of treatment or therapy, or at the start of the treatment or therapy, to assist in the analysis and evaluation of patient progress and/or response at one or more later points in time during the period that the patient is receiving treatment and undergoing clinical and medical evaluation.

In monitoring a patient's levels of one or more of the markers of the invention over a period of time, which may be days, weeks, months, and in some cases, years, or various intervals thereof, the patient's body fluid sample, e.g., a serum or plasma sample, is collected at intervals, as determined by the practitioner, such as a physician or clinician, to determine the levels of one or more of the markers in the cancer patient compared to the respective levels of one or more of these analytes in normal individuals over the course or treatment or disease. For example, patient samples can be taken and monitored every month, every two months, or combinations of one, two, or three month intervals according to the invention. Quarterly, or more frequent monitoring of patient samples, is advisable.

The levels of the one or more markers found in the patient are compared with the respective levels of the one or more of these markers in normal individuals, and with the patient's own marker levels, for example, obtained from prior testing periods, to determine treatment or disease progress or outcome. Accordingly, use of the patient's own marker levels monitored over time can provide, for comparison purposes, the patient's own values as an internal personal control for long-term monitoring of marker levels, and thus cancer presence and/or progression. As described herein, following a course of treatment or disease, the determination of an increase or a decrease in one or more of the marker levels in the cancer patient over time compared to the respective levels of one or more of these markers in normal individuals reflects the ability to determine the severity or stage of a patient's cancer, or the progress, or lack thereof, in the course or outcome of a patient's cancer therapy or treatment.

Increases or decreases in the levels of the markers in cancer patients are determined by comparing the values obtained from analyzing cancer patient samples compared to the normal control range expression levels. A biomarker is said to be over-expressed if expression of the marker is at least 2 fold greater in the cancer patient relative to a normal control, and a biomarker is said to be under expressed if the expression of the marker is at least 2 fold greater in the normal control relative to in the cancer patient.

In monitoring a patient over time, a reduction in the levels of one or more of a patient's marker levels from increased levels (i.e., at least 2 fold over-expressed) compared to normal range values to levels at or near to the levels of the analytes found in normal individuals is indicative of treatment progress or efficacy, and/or disease improvement, remission, tumor reduction or elimination, and the like. Likewise, in all of the methods described in the embodiments of this invention, a determination of a reduction of one or more of a patient's marker levels from an elevated level (i.e., at least 2 fold over-expressed) to, or approximately to, the respective levels of one or more of these analytes found in normal individuals provides a further aspect of the methods of the invention, in which a patient's improvement, recovery or remission, and/or treatment progress or efficacy, is able to be ascertained over time following performance of the method.

Another embodiment of the present invention encompasses a method of monitoring a cancer patient's course of disease, or the efficacy of a cancer patient's treatment or therapy. The patient's treatment or therapy can involve traditional therapies, such as hormone therapy, chemotherapeutic drug therapy, radiation, or novel therapies, or a combination of any of the foregoing. The method involves measuring levels of one or more markers in a body fluid sample of the cancer patient and determining if the levels of one or more of the markers in the patient's sample are changed by at least 2 fold compared to the respective levels of one or more of these analytes in normal controls during the course of disease or cancer treatment. In accordance with the method, a change in the levels of the marker in the cancer patient compared to the respective levels of the marker in normal controls is indicative of a change in stage, grade, severity or progression of the patient's cancer and/or a lack of efficacy or benefit of the cancer treatment or therapy provided to the patient during a course of treatment, e.g., poor treatment or clinical outcome.

As will be understood by the skilled practitioner in the art, the monitoring method according to this invention is preferably, performed in a serial or sequential fashion, using samples taken from a patient during the course of disease, or a disease treatment regimen, (e.g., after a number of days, weeks, months, or occasionally, years, or various multiples of these intervals) to allow a determination of disease progression or outcome, and/or treatment efficacy or outcome. If the sample is amenable to freezing or cold storage, the samples may be taken from a patient (or normal individual) and stored for a period of time prior to analysis.

In another of its embodiments, the present invention encompasses the determination of the amounts or levels of one or more additional cancer markers in conjunction with the determination of the levels of one or more of the markers of the invention in a sample to be analyzed.

The present invention also includes a method of assessing the efficacy of a test composition for inhibiting cancers, such as colon cancer. As described above, differential expression levels of the marker sequences of the invention correlate with the cancerous state of cancer cells, particularly colon cancer cells. It is recognized that changes in the expression levels of the marker sequences of the present invention result from the cancerous state of cells. Thus, composition which inhibit cancer in a patient will cause the expression levels of the marker sequences to change to a level near the normal level of expression for the marker sequences. The method thus comprises comparing expression levels of one or more marker sequences in a first biological sample maintained in the presence of a test composition with those of the same marker sequences in a second biological sample maintained in the absence of the test composition. A significant difference in the expression levels of one or more marker sequences is an indication that the test composition inhibits the cancer. In a preferred embodiment, the cancer is colon cancer, and the marker sequences are the ones listed in Tables 1 and 2. In another embodiment, the cell samples may be aliquots of a single sample obtained from either a healthy subject or a patient with cancerous conditions.

XIV Modulators of the Marker Sequences

It is recognized that changes in the expression levels of the marker sequences likely induce, maintain, and promote the cancerous state of cells. Thus, another aspect of the present invention is directed to the modulators of the marker sequences capable of modulating the differentiation and proliferation of cells. In this regard, the present invention provides assays for determining compounds that modulate the expression of the marker sequences. The compounds can be used to modulate the biological activity of the polypeptides encoded by the marker sequences or the marker sequences themselves. Compounds can also be useful in a variety of different environments, including as medicinal agents to treat or prevent disorders associated with cancer.

Methods of identifying compounds generally comprise steps in which a compound is placed in contact with a marker sequence, its transcription product, its translation product, or other target, and determination of whether the compound modulates the marker sequence. For modulating the expression of a marker sequence, a method can comprise, in any effective order, one or more of the following steps, e.g., contacting the marker sequence (e.g., in a cell population) with a test compound under conditions effective for said test compound to modulate the expression of the marker sequence, and determining whether said test agent modulates said sequence. A compound can modulate expression of a sequence at any level, including transcription (e.g., by modulating the promoter), translation, and/or perdurance of the nucleic acid (e.g., degradation, stability, etc.) in the cell.

For modulating the biological activity of polypeptides, a method can comprise, in any effective order, one or more of the following steps, e.g. , contacting a polypeptide (e.g., in a cell, lysate, or isolated) with a test compound under conditions effective for said test agent to modulate the biological activity of said polypeptide, and determining whether said test compound modulates said biological activity.

Contacting the polynucleotide or polypeptide with the test compound can be accomplished by any suitable method and/or means that places the compound in a position to functionally control expression or biological activity of the gene or its product in the sample. Functional control indicates that the compound can exert its physiological effect through whatever mechanism it works. The choice of the method and/or means can depend upon the nature of the compound and the condition and type of environment in which the gene or its product is presented, e.g., lysate, isolated, or in a cell population (such as, in vivo, in vitro, organ explants, etc.). For example, if the cell population is an in vitro cell culture, the compound can be contacted with the cells by adding it directly into the culture medium. If the compound cannot dissolve readily in an aqueous medium, it can be incorporated into liposomes, or another lipophilic carrier, and then administered to the cell culture. Contact can also be facilitated by incorporation of compound with carriers and delivery molecules and complexes, by injection, by infusion, etc.

After the agent has been administered in such a way that it can gain access to the gene or gene product (including DNA, mRNA, and polypeptides), it can be determined whether the test compound modulates its expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test compound has-an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the compound. This effect includes, increase, decrease, block, inhibit, enhance, etc.

A test compound can be of any molecular composition, e.g., chemical compounds, biomolecules, such as polypeptides, lipids, nucleic acids (e.g., antisense to a polynucleotide) carbohydrates, antibodies, ribozymes, double-stranded RNA, aptamers, etc. For example, if a polypeptide to be modulated is a cell-surface molecule, a test compound can be an antibody that specifically recognizes it and, e.g., causes the polypeptide to be internalized, leading to its down regulation on the surface of the cell. Such effect does not have to be permanent, but can require the presence of the antibody to continue the down-regulatory effect. Antibodies can also be used to modulate the biological activity of a polypeptide in a lysate or other cell-free form.

XV Drug Screening

In one aspect, the present invention is also directed to methods for screening drugs that inhibit cancer, particularly colon cancer. Drug screening is performed by adding a test compound to a sample of cells, and monitoring the effect. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. Differences between treated and untreated cells indicates effects attributable to the test compound.

Desirable effects of a test compound include an effect on any phenotype that was conferred by the cancer-associated marker nucleic acid sequence. Examples include a test compound that limits the overabundance of mRNA, limits production of the encoded protein, or limits the functional effect of the protein. The effect of the test compound would be apparent when comparing results between treated and untreated cells. For example, candidate compounds may be identified that down-regulate expression of one specific gene. In one embodiment, candidate compounds may be identified that up-regulate expression of one specific gene. Generally a plurality of assay mixtures are run in parallel with different compound concentrations to obtain a differential response to the various concentrations. Typically, one of these concentrations serves as a negative control, i.e., at zero concentration or below the level of detection.

Screening assays can be based upon any of a variety of techniques readily available and known to one of ordinary skill in the art. In general, the screening assays involve contacting a cancerous cell (preferably a cancerous colon cell) with a candidate agent, and assessing the effect upon biological activity of a differentially expressed gene product. The effect upon a biological activity can be detected by, for example, detection of expression of a gene product of a differentially expressed gene (e.g., a decrease in mRNA or polypeptide levels, would in turn cause a decrease in biological activity of the gene product). Alternatively or in addition, the effect of the candidate agent can be assessed by examining the effect of the candidate agent in a functional assay. For example, where the differentially expressed gene product is an enzyme, then the effect upon biological activity can be assessed by detecting a level of enzymatic activity associated with the differentially expressed gene product. The functional assay will be selected according to the differentially expressed gene product.

The screening methods may include both in vitro and in vivo screening of a cell or tissue. One particular embodiment of in vitro method comprises a method of determining the efficacy of a test compound for inhibiting cancer in a subject, the method comprising comparing a) the expression level of one or more nucleic acid sequences in a first biological sample from the subject wherein the sample has been exposed to the test compound, with b) the expression level of said nucleic acid sequences in a second biological sample from the subject wherein the sample has not been exposed to the test compound, said nucleic acid sequences comprising one or more nucleic acid sequences selected from the group consisting of SEQ ID NOs: 1-93, wherein a change of at least two fold in the expression level of said nucleic acid sequences is an indication that the test compound is efficacious for inhibiting cancer in the subject.

In another embodiment, the in vivo methods of screening for compounds that alter the expression of the marker sequences comprise exposing a subject, preferably a mammal having cancer cells in which the marker sequences (either at mRNA or polypeptide level) are detectable, to a compound, and determining the level of the marker sequences. Where the differentially expressed gene is increased in expression in a cancerous cell, the compound of interest is those that decrease activity of the differentially expressed gene product, and where the differentially expressed gene is decreased in expression in a cancerous cell, the compound of interest is those that increase activity of the differentially expressed gene product.

Assays for determining the differentially expressed marker sequences (described supra) can be readily adapted in the screening assay embodiments of the present invention. Exemplary assays useful in screening candidate compounds include, but are not limited to, hybridization-based assays (e.g. use of nucleic acid probes or primers to assess expression levels), antibody-based assays (e.g. to assess levels of polypeptide gene products), binding assays (e.g. to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like.

In one embodiment, the candidate compound is naturally occurring or modified proteins. In another embodiment, candidate compounds are peptides. The peptides may be digests of naturally occurring proteins, or the one made by chemical synthesis. Furthermore, the synthetic process can be designed to generate randomized proteins, to allow the formation of all or most of the possible combinations over the length of the sequence, thus forming a library of randomized candidate proteinaceous drugs.

In another embodiment, the candidate compounds are nucleic acids, either naturally occurring or modified. In a preferred embodiment, the nucleic acid compounds are antisense nucleic acids. Drug candidates that are antisense molecules include antisense or sense oligonucleotides comprising a single-strand nucleic acid sequence (either RNA or DNA) capable of binding to target mRNA or DNA sequences for lung cancer molecules identified by the methods of the invention.

In yet another preferred embodiment, drug candidates are antibodies. An antibody used in methods for screening for a candidate drug may either bind a full length protein or a fragment thereof. In a preferred embodiment, the antibody binds a unique epitope on a target protein and shows little or no cross-reactivity. The term “antibody” is understood to include antibody fragments, as are known in the art, including Fab, Fab₂, single chain antibodies (Fv for example), chimeric antibodies, etc., either produced by the modification of whole antibodies or those synthesized de novo using recombinant DNA technologies known in the art. Antibodies as used herein as drug candidates include both polyclonal and monoclonal antibodies. Polyclonal antibodies can be raised in a mammal, for example, by one or more injections of an antigenic agent and, if desired, an adjuvant. It may be useful to conjugate the antigenic agent to a protein known to be immunogenic in the mammal being immunized.

In yet another embodiment, the candidate compounds are chemical compounds. In a preferred embodiment, the candidate compounds are small organic compounds having a molecular weight of more than 100 and less than about 2500 daltons. Candidate compounds may also include functional groups necessary for structural interaction with proteins or nucleic acids.

XVI Kits

The present invention also provides for kits that contain the necessary reagents for detection of the expression levels (either at RNA or polypeptide level) of the individual and/or combinations of marker sequences in a biological sample. Reagents can include marker sequence-specific probes/primers and antibodies as described supra. Kits can also contain a control/reference value or a set of control/reference values indicating normal and various clinical progression stages of cancer. In a preferred embodiment, the control/reference value or a set of control/reference values are indicative of normal and various clinical progression stages of colon cancer. Moreover, kits can contain positive controls, and/or negative controls for comparison with the test sample. A negative control can contain a sample that does not have any marker RNA or polypeptide. A positive control can contain a sample that have various known levels of marker RNA or polypeptide. Kits can also contain any combinations of the marker sequence-specific probes/primers and/or antibodies. Kits can also contain instructions for conducting the assays and for interpreting the results. For antibody-based kit, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label. For oligonucleotide-based kits, the kit can comprise, for example: (1) an oligonucleotide, e.g., a detectably labeled oligonucleotide, which hybridizes to a nucleic acid sequence encoding a polypeptide corresponding to a marker sequence of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

Such kits can be used to determine whether a subject is suffering from or at an increased risk of developing cancer, particularly colon cancer. Furthermore, such kits can be used to determine the prognosis, stage, or monitoring the progression of cancer, particularly colon cancer. Furthermore, such kits can be used for drug screening or for selection of treatment for cancer, particularly colon cancer.

### EXAMPLES

The examples below are non-limiting and are merely representative of various aspects and features of the present invention.

### Example 1

### Identification of Differentially Expressed Marker Sequences

Twenty well characterized, microdissected samples of colorectal cancer tissue were obtained from consenting patients. A second set of twenty, microdissected samples of normal adjacent colon tissue were also obtained. Total RNA was extracted from these samples using RNeasy kits (QIAGEN, Valencia, Calif.) according to the manufacturer's instructions. Expression profiling was performed using the GeneChip expression arrays from Affymetrix (Santa Clara, Calif.). Reverse transcription, second-strand synthesis, and probe generation was accomplished by standard Affymetrix protocols. The Human Genome U133A GeneChip, which contains more than 15,000 substantiated human genes, was hybridized, washed, and scanned according to Affymetrix protocols. Changes in cellular mRNA levels in the cancerous tissues were compared with mRNA levels in the normal colon tissues. GeneSpring v4.2 (Silicon Genetics, Redwood City, Calif.) was used to normalize and scale results and compare gene expression levels in the cancer tissue relative to that in the normal tissue.

Applying a set of filters to the normalized data identified the up- and down-regulated genes. First, a non-parametric test defined the genes that were statistically associated with either the cancer or the normal samples. Next, a pair of filters was used to remove the genes with low signals and to set a high threshold for a minimum expression levels. The final filter required a three-fold average expression difference between the two conditions (cancer and normal).

This analysis resulted in 47 genes that were up-regulated in the colorectal cancer tissue relative to the normal adjacent colon tissue. These genes are identified in Table 1. Likewise, 46 down-regulated genes were identified in the colorectal cancer tissue relative to the normal adjacent colon tissue. These genes are listed in Table 2.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of detecting differential expression of one or more nucleic acid sequences in a biological sample, comprising:
(a) obtaining the sample from a subject; and
(b) detecting a change in the expression level of one or more nucleic acid sequences relative to a control expression level of the nucleic acid sequences, said nucleic acid sequences comprising one or more nucleic acid sequences selected from the group consisting of SEQ ID NOs: 1-93.

2. The method of claim 1, wherein said step of detecting comprises:
(a) contacting said sample with a polynucleotide probe comprising at least 12 consecutive nucleotides of a nucleic acid sequence, said probe is capable of hybridizing under stringent conditions to a nucleic acid sequence selected from the group consisting of SEQ ID NOs: 1-93;
(b) detecting the hybridization of said polynucleotide probe to said nucleic acid sequence selected from the group consisting of SEQ ID NOs: 1-93, wherein the signal intensity of hybridization is indicative of the expression level of a nucleic acid sequence selected from the group consisting of SEQ ID NOs: 1-93.

3. The method of claim 2, wherein said probe comprises a detectable label.

4. The method of claim 1, wherein said change in the expression level is either an increase or an decrease in expression level.

5. The method of claim 1, wherein said change in the expression level is at least two fold.

6. A method of detecting cancer or a pre-malignant condition thereof in a subject comprising comparing a) the expression level of one or more nucleic acid sequences in a biological sample from the subject with b) a control expression level of said nucleic acid sequences, said nucleic acid sequences comprising one or more nucleic acid sequences selected from the group consisting of SEQ ID NOs: 1-93, wherein a change of at least two-fold in the expression level of said nucleic acid sequences is indicative of cancer or pre-malignant condition.

7. The method of claim 6, wherein said change in the expression level is either an increase or decrease in the expression level.

8. A method of monitoring the onset, progression, or regression of cancer or a pre-malignant condition thereof in a subject, the method comprising:
(a) detecting in a biological sample of the subject at a first point in time, the expression of one or more nucleic acid sequences comprising one or more nucleic acid sequences selected from the group consisting of SEQ ID NOs: 1-93;
(b) repeating step (a) at a subsequent point in time; and
(c) comparing the expression level detected in steps (a) and (b), wherein a change in the expression level is indicative of progression of cancer or a pre-malignant condition thereof in the subject.

9. The method of claim 8, wherein the change in the expression level is either an increase or decrease.

10. A method of determining prognosis for cancer or a pre-malignant condition thereof in a subject, comprising:
(a) detecting in a biological sample of the subject, the expression level of one or more nucleic acid sequences comprising one or more nucleic acid sequences selected from the group consisting of SEQ ID NOs: 1-93;
(b) comparing the expression level detected in steps (a) with a reference expression level of said nucleic acid sequences; and
(c) evaluating the prognosis of the subject based on the comparison in step (b).

11. The method of claim 10, wherein the reference expression level is the expression level of said nucleic acid sequences in cancer free or normal sample.

12. The method of claim 10, wherein the reference expression level is the expression level of said nucleic acid sequences cancer samples that are known not to progress to aggressive form.

13. A method of determining the efficacy of a test compound for inhibiting cancer in a subject, the method comprising comparing a) the expression level of one or more nucleic acid sequences in a first biological sample from the subject wherein the sample has been exposed to the test compound, with b) the expression level of said nucleic acid sequences in a second biological sample from the subject wherein the sample has not been exposed to the test compound, said nucleic acid sequences comprising one or more nucleic acid sequences selected from the group consisting of SEQ ID NOs: 1-93, wherein a change of at least two fold in the expression level of said nucleic acid sequences is an indication that the test compound is efficacious for inhibiting cancer in the subject.

14. The method of claim 13, wherein the change in the expression level is either an increase or decrease.

15. A method of determining the efficacy of a therapy for inhibiting cancer in a subject, the method comprising comparing a) the expression level of one or more nucleic acid sequences in a first biological sample from the subject prior to providing at least a portion of the therapy to the subject, with b) the expression level of said nucleic acid sequences in a second biological sample from the subject following the provision of the portion of the therapy, said nucleic acid sequences comprising one or more nucleic acid sequences selected from the group consisting of SEQ ID NOs: 1-93, wherein a change of at least two fold in the expression level of said nucleic acid sequences is an indication that the therapy is efficacious for inhibiting cancer in the subject.

16. The method of claim 15, wherein the change in the expression level is either an increase or decrease.

17. A method of selecting a composition for inhibiting cancer in a subject, the method comprising:
(a) obtaining a first biological sample comprising cancer cells from the subject;
(b) separately exposing aliquots of the sample in the presence of a plurality of test compositions;
(c) comparing the expression level of one or more nucleic acid sequences in each of the aliquots from (b) with the expression level in the sample produced by (a), said nucleic acid sequences comprising one or more nucleic acid sequences selected from the group consisting of SEQ ID NOs: 1-93; and
(d) selecting one of the test compositions which induces a change of at least two fold in the expression level of said nucleic acid sequences in one aliquot containing the test composition.

18. The method of claim 17, wherein the change in the expression level is either an increase or decrease.

19. A method of inhibiting cancer in a subject, the method comprising:
(a) obtaining a first biological sample comprising cells from the subject;
(b) administering to the subject one or more test compositions;
(c) obtaining a second biological sample comprising cells from the subject of (b); and
(d) comparing the expression level of one or more nucleic acid sequences in the first sample with the expression level of said nucleic acid sequences in the second sample, wherein a change of at least two fold in the expression level is indicative of inhibition of cancer by said test compositions.

20. A polypeptide comprising a polypeptide sequence selected from the group consisting of SEQ ID NOs: 94-186.

21. An antibody that specifically binds to a polypeptide sequence selected from the group consisting of SEQ ID NOs: 94-186.

22. The antibody of claim 21, wherein said antibody is polyclonal antibody.

23. The antibody of claim 21, wherein said antibody is monoclonal antibody.

24. A method of detecting in a biological sample the presence of a polypeptide comprising a polypeptide sequence selected from the group consisting of SEQ ID NOs: 94-186, said method comprising:
(a) obtaining said biological sample from a subject;
(b) contacting said sample with a polypeptide ligand which is capable of binding to one or more of SEQ ID NOs: 94-186; and
(c) detecting the binding of said polypeptide ligand to said polypeptide, wherein detecting of binding is indicative of the presence of said polypeptide sequence comprising a polypeptide sequence selected from the group consisting of SEQ ID NOs: 94-186 in said biological sample.

25. The method of claim 24, wherein the polypeptide ligand is an antibody.

26. The method of claim 24, wherein the polypeptide ligand comprises a detectable label.

27. The method of claim 25, wherein the antibody is a monoclonal antibody.

28. A method of detecting cancer or a pre-malignant condition thereof in a subject comprising:
(a) obtaining a biological sample from a subject;
(b) contacting the sample with one or more polypeptide ligands that bind specifically to one or more polypeptides comprising a polypeptide sequence selected from the group consisting of SEQ ID NOs: 94-186;
(c) determining specific binding; and
(d) comparing the specific binding between the polypeptide ligands and the polypeptides in the sample with the specific binding between the polypeptide ligands and the polypeptides in a cancer-free sample, wherein a significant change in the specific binding is diagnostic for cancer in the subject.

29. A method of monitoring the onset, progression, or regression of cancer in a subject, comprising:
(a) contacting at a first point in time a first biological sample with one or more polypeptide ligands that specifically bind to one or more polypeptides comprising a polypeptide sequence selected from the group consisting of SEQ ID NOs: 94-186, determining specific binding between the polypeptide ligands and the polypeptides;
(b) contacting at a subsequent point in time a second biological sample with said polypeptide ligands that specifically bind to one or more polypeptides comprising a polypeptide sequence selected from the group consisting of SEQ ID NOs: 94-186, determining specific binding between the polypeptide ligands and the polypeptides; and
(c) comparing the specific binding in the first biological sample to the specific binding in the second biological sample, wherein a significant change in the specific binding is an indication of the onset, progression, or regression of cancer.

30. A method of determining prognosis for cancer or a pre-malignant condition thereof in a subject, comprising:
(a) contacting a biological sample obtained from a subject having cancer with one or more polypeptide ligands that bind specifically to one or more polypeptides comprising a polypeptide sequence selected from the group consisting of SEQ ID NOs: 94-186;
(b) determining specific binding;
(c) comparing the specific binding between the polypeptide ligands and the polypeptides in the sample with the specific binding between the polypeptide ligands and the polypeptides either in a cancer-free sample or in a cancer sample that is known not to progress to aggressive form; and
(d) evaluating the prognosis of the subject based on the comparison in step (c).

31. A method of determining the efficacy of a test compound for inhibiting cancer in a subject, the method comprising comparing a) in a first biological sample from the subject binding between one or more polypeptide ligands that specifically bind to one or more polypeptides comprising a polypeptide sequence selected from the group consisting of SEQ ID NOs: 94-186 and one or more polypeptides comprising a polypeptide sequence selected from the group consisting of SEQ ID NOs: 94-186, wherein the sample has not been exposed to the test compound, with b) in a second biological sample from the subject, the specific binding of said polypeptide ligands and said polypeptides, wherein the sample has been exposed to the test compound, and wherein a significant change in the specific binding is an indication that the test compound is efficacious for inhibiting cancer in the subject.

32. A method of determining the efficacy of a therapy for inhibiting cancer in a subject, comprising comparing a) in a first biological sample from the subject prior to a treatment, binding between one or more polypeptide ligands that specifically bind to one or more polypeptides comprising a polypeptide sequence selected from the group consisting of SEQ ID NOs: 94-186 and one or more polypeptides comprising a polypeptide sequence selected from the group consisting of SEQ ID NOs: 94-186, with b) in a second biological sample from the subject following the treatment, the specific binding of said polypeptide ligands and said polypeptides, and wherein a significant change in the specific binding is an indication that the test compound is efficacious for inhibiting cancer in the subject.

33. A method of selecting a composition for inhibiting cancer in a subject, comprising
(a) obtaining a first biological sample comprising cancer cells from the subject;
(b) separately exposing aliquots of the sample in the presence of a plurality of test compositions;
(c) comparing the specific binding between one or more polypeptide ligands and one or more polypeptides in each of the aliquots from (b) with the specific binding between said polypeptide ligands and said polypeptides in each of the aliquots from (a), wherein said ligands comprising a polypeptide sequence selected from the group consisting of SEQ ID NOs: 94-186, and wherein said polypeptides comprising a polypeptide sequence selected from the group consisting of SEQ ID NOs: 94-186; and
(d) selecting one of the test compositions which induces a significant change in specific binding.

34. A method of inhibiting cancer in a subject with cancer, comprising:
(a) obtaining a first biological sample comprising cells from the subject;
(b) administering to the subject one or more test compositions;
(c) obtaining a second biological sample comprising cells from the subject of (b); and
(d) comparing the specific binding between one or more polypeptide ligands and one or more polypeptides in the first sample with the specific binding between said polypeptide ligands and said polypeptides in the second sample, wherein said ligands comprising a polypeptide sequence selected from the group consisting of SEQ ID NOs: 94-186, and wherein said polypeptides comprising a polypeptide sequence selected from the group consisting of SEQ ID NOs: 94-186, and wherein a significant change in the specific binding is an indication of inhibition cancer by said test compositions.
